(19) United States
(12) Patent Application Publication
(10) Pub. No.: US 2021/0370205 A1
(43) Pub. Date: Dec. 2, 2021

(54) REMOVAL OF WATER CONTAMINANTS USING ENHANCED CERAMIC FILTRATION MATERIALS

(71) Applicant: UWM Research Foundation, Inc., Milwaukee, WI (US)

(72) Inventors: Yin Wang, Whitefish Bay, WI (US); Shangping Xu, Mequon, WI (US); Haiyan Yang, Milwaukee, WI (US); John Bender, Milwaukee, WI (US)

(21) Appl. No.: 17/286,124

(22) PCT Filed: Oct. 17, 2019
(86) PCT No.: PCT/US2019/056674

§ 371 Date: Apr. 16, 2021

Related U.S. Application Data
(60) Provisional application No. 62/746,724, filed on Oct. 17, 2018.

(51) Int. Cl. B01D 39/06; C04B 38/06; C04B 33/04; C04B 33/32; C04B 33/13; C04B 38/00; A61L 2/02; B01D 39/20; C02F 1/00

(52) U.S. Cl. CPC B01D 39/06; C02F 2101/103; C04B 33/04; C04B 33/32; C04B 33/1305; C04B 38/0051; A61L 2/022; B01D 39/2082; C02F 1/001; C04B 2235/349; B01D 2239/0407; B01D 2239/0442; B01D 2239/10; B01D 2239/1216; B01D 2239/1233; B01D 2239/0283; B01D 2239/1208; B01D 2239/125; C04B 38/0675

(57) ABSTRACT

A filter material composing a ceramic clay having an interconnected network of pores formed from cellulose fiber combustion is useful for removing chemical and biological contaminants from a water supply. Coating the ceramic clay with lanthanum enhances the removal of anionic species of As(V), As(III), Cr(VI), microbes and virus.

### CROSS-REFERENCE TO RELATED APPLICATIONS

This application claims priority to U.S. Provisional Application No. 62/746,724, filed Oct. 17, 2018, which is hereby incorporated by reference in its entirety.

### STATEMENT OF GOVERNMENT INTEREST

This invention was made with government support under grant number 1540032 awarded by the National Science Foundation. The government has certain rights in the invention.

### TECHNICAL FIELD

The present disclosure relates to enhanced ceramic filtration materials with applications in removal of water contaminants such as anions and microbes.

### BACKGROUND

Due to the high toxicity and ubiquitous presence in the environment, arsenic contamination in drinking water is one of the greatest threats to public health. About 1.8 billion people, most of whom live in developing countries, do not have the access to safe drinking water and the consumption of unsafe drinking water can lead to a wide variety of diseases. For instance, it was estimated that ˜50 million people in Asia are exposed to arsenic (As) levels exceeding 50 μg/L and half million out of the 50 million people will die from As related cancers. Additionally, at least four million people are exposed to high concentrations of As in drinking-water, primarily rural dwellers consuming water from wells in Latin America. In addition to As, chromium (Cr) is among the most widespread heavy metal pollutants in groundwater, because of the improper disposal of industrial wastes and dissolution of Cr-containing minerals. According to World Health Organization (WHO), improved drinking water supply alone can reduce the global disease burden by 4%. The development of effective, low-cost, low-maintenance and environmentally friendly water filtration techniques can have far-reaching public health, social, and economic benefits. Thus, it is imperative to develop effective, affordable, and low-maintenance technologies appropriate for point-of-use (POU) applications for household water treatment.

Among various arsenic and chromate treatment technologies, adsorption-based filtration is most attractive for POU water treatment because it is easy to operate, highly robust, and effective. Adsorption represents a mainline strategy in the removal of chemical and microbial contaminants from drinking water. Recent interests have focused on the development of adsorbents from naturally abundant and/or reusable materials. For example, natural minerals such as zeolite usually carry negative surface charges and display high cation exchange capacity, and thus they have been widely used as an inexpensive and yet effective adsorbent for the removal of positively charged contaminants such as heavy metals (e.g., Cd²⁺, Pb²⁺) from water. The negative charges of natural minerals, however, make them generally ineffective in the adsorption of anionic contaminants, such as arsenic (As(V), As(III)) and chromate (Cr(VI)).

Ceramic materials have gained increasing attention during the past decade for water filtration applications. Porous ceramic materials are generally prepared with the use of earth-abundant clay minerals as substrates and organic wastes as pore forming materials (e.g., sawdust, rice husk, flour), and can be fabricated into various shapes (e.g., granule, disk, powder, tube, candle, and pot filters). The low-cost and easy-to-use feature makes ceramic-based water filtration a promising and sustainable treatment technique. Such filters, however, are ineffective for the removal of arsenic, because of the low affinity between ceramic surface and arsenic. The development of iron (hydr)oxide-modified ceramic material was reported recently, which exhibited enhanced As(V) removal capacity up to ˜7 mg/g, while the removal efficiency of As(III) and Cr(VI) were not examined.

Further, silver impregnation has been commonly applied to ceramic filters because of the antimicrobial properties of silver. However, its relatively high cost and short filter service life motivate the investigation of silver-free strategies to improve the performance of ceramic filters. So far, physical filtration (size exclusion and straining) is considered the primary mechanism for microbial removal by ceramic water filters. Several previous studies reported the important role of pore properties in the flow rate and bacteria removal of ceramic filters. Notably, pores of ceramic filters are created due to the firing of combustible material in the filter fabrication process, and proper size and ratio of the combustible material have been identified among the key design parameters for an effective ceramic filter. Generally, the porosity of ceramic filters can be tuned in the range of 0.2-0.5 by adjusting the size and ratio of the combustible material. However, varied performances have been observed for ceramic filters with similar porosity, indicating that pore properties (e.g., pore size distribution) other than porosity may also be crucial in controlling the filter flow rate and microbial reduction. A previous study found that filters made of redart clay had 75% of their pores with diameters <5 μm and filters with smaller pores caused higher bacteria retention. However, the relationship among pore size distribution, flow rate, and bacterial removal is still insufficiently understood. Because of the low cost, high porosity, and stable chemical property, ceramic material represents a promising support medium with high potential to be further improved toward more efficient removal of As(V), As(III), Cr(VI), and microbes.

Lanthanum (La) is an abundant rare earth element and is widely used for phosphate abatement. Recently, a few pioneering studies reported that La-modified metal oxides significantly improved As(V) removal capacity because of the strong interactions between La and As(V). Because of the environmentally benign and relatively inexpensive nature of La, La coating for ceramic materials may represent a promising arsenic removal strategy for POU applications. However, the effectiveness of La-amended ceramic materials as filtration media for As(V), As(III), and Cr(VI) removal remains unexplored, and the mechanisms that govern the removal of As(V), As(I), and Cr(VI) are still insufficiently understood.

### SUMMARY

Disclosed herein are ceramic filtration materials and devices for removal of contaminants from a water supply. (e.g., As(V), As(III), Cr(VI), bacteria, and viruses).

In one aspect, the invention provides a filter material comprising a ceramic clay having an outer surface and a network of pores, the network of pores having a shape and a volume defined by combustion of cellulose fibers in a mixture of the cellulose fibers and a raw clay material. In another aspect, the invention provides a method of preparing the filter material comprising (a) homogenizing a mixture of raw clay material, cellulose fibers, and water; (b) drying the homogenized mixture; and (c) firing the dried, homogenized mixture so as to incinerate the cellulose fibers. In another aspect, the invention provides a filter material prepared by the foregoing method. In yet another aspect, the invention provides a filter device comprising the filter material and a housing. In still another aspect, the invention provides a method of removing a water contaminant from a water supply comprising contacting the water supply with the filter device, as described herein, to remove the water contaminant.

In another aspect, the invention provides a filter material comprising (a) a ceramic clay having an outer surface and a network of pores, the network of pores having a shape and a volume defined by combustion of cellulose fibers in a mixture of the cellulose fibers and a raw clay material; and (b) a lanthanum-containing coating disposed on the outer surface and within the network of pores of the ceramic clay. In another aspect, the invention provides a method of preparing the filter material comprising (a) treating an uncoated ceramic clay, as described herein, with a solution containing one or more lanthanum salts selected from the group consisting of La(NO₃)₃, LaCl₃, LaBr₃, LaI₃, LaF₃, La₂(SO₄)₃, LaPO₄, La₂(C₂O₄)₃, La₂O₃, LaOOH, La(OH)₃, La₂S₃, La(CH₃CO₂)₃, and LaAlO₃; and (b) heating the treated ceramic clay at 100° C. to 800° C. In another aspect, the invention provides a filter material prepared by the foregoing method. In yet another aspect, the invention provides a filter device comprising the filter material and a housing. In still another aspect, the invention provides a method of removing a water contaminant from a water supply comprising contacting the water supply with the filter device, as described herein, to remove the water contaminant.

In one aspect, the invention provides La-coated ceramic materials prepared by utilizing lanthanum nitrate as an additive to modify the surface of granular ceramic sorbents that were made from natural clay and recycled paper fiber (cellulose fiber). In particular, ceramic materials with La coating were fabricated into two prototype filters (e.g., ceramic granules-packed column filter and ceramic disk filter) for the removal of As(V), As(III), and Cr(VI).

Another aspect of the invention provides a method of preparing unmodified ceramic filters with the use of cellulose fibers to create various pore size distributions in the ceramic filters, where pore size distribution affects the flow rate and bacterial removal of the ceramic filter.

### BRIEF DESCRIPTION OF THE DRAWINGS

FIG. 1. Schematic diagram of (a) ceramic granules filter and (b) ceramic disk filter for arsenic removal experiments.

FIG. 2. Geometric illustration of porous ceramic filter modeled as layers of bundle of parallel capillaries: (a) “vertical” cross section of the ceramic filter in plane (x,z); (b) cross section of the geometric model in plane (x,y).

FIG. 3. As(V) sorption amount with and without vacuum treatment during La coating process. Initial As(V) concentrations are 67 mg/L. Contact time is 24 h. Adsorbent dosage is 0.5 g/L. DE means diatomaceous earth, kao means kaolinite, and the numbers in the parenthesis means the percentage of combustible materials (e.g., cellulose fiber).

FIG. 4. As(III) sorption amount with and without vacuum treatment during La coating process. Initial As(III) concentrations are 35 mg/L. Contact time is 24 h. Adsorbent dosage is 0.5 g/L. DE means diatomaceous earth, kao means kaolinite, and the numbers in the parenthesis means the percentage of combustible materials (e.g., cellulose fiber).

FIG. 5. (a) Evaluation of As(V) and Cr(VI) adsorption on granular ceramic material modified with La(NO₃)₃ at different firing temperatures. Initial As(V) concentration and adsorbent dosage were 30 mg·L⁻¹ and 1.0 g·L⁻¹, and initial Cr(VI) concentration and adsorbent dosage are 10 mg·L⁻¹ and 0.5 g·L⁻¹. Error bars represent standard deviations from triplicate experiments (n=3). (b) Proposed lanthanum species present at different firing temperatures of granular ceramic material modified with La(NO₃)₃.

FIG. 6. Effects of ionic strength and coexisting ions on As(V) and Cr(VI) adsorption. Effect of (a) ionic strength in NaCl background solution and (b) coexisting anions (1 mM) on As(V) and Cr(VI) adsorption on La-modified ceramic granules. Initial As(V) concentration and adsorbent dosage were 20 mg/L and 1.0 g/L, and initial Cr(VI) concentration and adsorbent dosage are 3 mg/L and 0.5 g/L, solution pH is 6.0 adjusted by 0.1 mol/L HCl.

FIG. 7. Adsorption kinetics for (a) As(V) and (b) Cr(VI) on La-modified granular ceramic materials treated at 385° C. Initial As (V) concentration and adsorbent dosage are 20 mg·L⁻¹ and 1.0 g·L⁻¹, and initial Cr (VI) concentration and adsorbent dosage are 3 mg·L⁻¹ and 0.5 g·L₋₁. Error bars represent standard deviations from triplicate experiments (n=3).

FIG. 8. Intra-particle diffusion model fit of As(V) and Cr(VI) adsorption onto La-modified granular ceramic materials.

FIG. 9. Adsorption isotherms for (a) As(V) and (b) Cr(VI) on La-modified granular ceramic materials treated at 385° C. The contact time is 24 h, and adsorbent dosages are 1.0 g·L⁻¹ for As(V) and 0.5 g·L⁻¹ for Cr(VI). Error bars represent standard deviations from triplicate experiments (n=3).

FIG. 10. SEM images of granular ceramic materials (a) without and with La modification at (b) 300, (c) 385, (d) 500 and (e) 800° C.

FIG. 11. TGA of (a) La(NO₃)₃, (b) ceramic granules mixed with La(NO₃)₃, and (c) ceramic granules alone.

FIG. 12. Fractions of (a) As(V) species and (b) Cr (VI) species at different pH in water.

FIG. 13. Zeta potential as a function of pH for La-modified ceramic materials treated at different firing temperatures.

FIG. 14. FTIR spectra of La-modified ceramic materials treated at different firing temperatures.

FIG. 15. SEM images of (a) uncoated and (b) La-coated ceramic material. Insets show high-resolution images of the ceramic surface.

FIG. 16. XRD patterns of uncoated and La-coated ceramic materials. The peaks associated with reference patterns of quartz (PDF #00-005-0490) and illite (PDF #00-009-0343) are labeled.

FIG. 17. Effluent arsenic concentrations in the bench-scale prototype La-coated ceramic granules and disk filters. Influent As(V) and As(III) concentrations are 0.120 and 0.125 mg/L, respectively.

FIG. 18. Adsorption kinetics (a,b) and isotherms (c,d) for As(V) and As(III) on La-coated ceramic materials in 292.5 mg/L (5 mM) NaCl at pH 6.0. Initial As(V) and As(III) concentrations for kinetics experiments are 5 mg/L. Sorbent dosages for both kinetics and isotherm experiments are 1.0 g/L.

FIG. 19. Effect of initial pH on As(V) and As(III) sorption by La-coated ceramic materials. Initial As(V) and As(III) concentration are 20 and 5 mg/L, respectively, and sorbent dosage is 1.0 g/L.

FIG. 20. As(V) and As(III) removal by the La-coated ceramic material in the presence of coexisting anions at pH 6. Concentrations of coexisting anions were 5 mM for chloride, nitrate, sulfate and carbonate, 1 mM for silicate, and 0.1 mM for phosphate (the highest level examined in Table 1). Initial As(V) and As(III) concentrations were 0.120 mg/L, and the sorbent dosage was 1.0 g/L.

FIG. 21. Effect of coexisting anions on (a) As(V) and (b) As(III) adsorption by La-coated ceramic granules. Initial As(V) and As(III) concentration 20 mg/L and 5 mg/L, adsorbent dose 1.0 g/L.

FIG. 22. FTIR spectra (a), zeta potential (b) and XPS As 3d spectra (c) of pristine ceramic material, and La-coated ceramic material before and after As(V) and As(III) sorption.

FIG. 23. Fractions of (a) As (III) and (b) As (V) species at different pH.

FIG. 24. XPS La 3d5/2 spectra of La-coated ceramic material before and after As(V) and As(III) sorption.

FIG. 25. SEM images (a-c) and mercury incremental intrusion curves (d-f) of ceramic filters made of different combustible materials.

FIG. 26. SEM images and size distribution of (a) cellulose fiber, (b) rice husk and (c) starch used as combustible material in ceramic filters fabrication. Cellulose fiber, starch and rice husk were simplified in tubular, spherical and cubic shapes, and the fiber diameter, sphere diameter and cube width were measured for size distribution analysis, respectively.

FIG. 27. FTIR spectra of ceramic filters made of different combustible materials.

FIG. 28. Average flow rate of ceramic filters made of different combustible materials in a series of ratios.

FIG. 29. Log reduction values (LRVs) for E. coli by ceramic filters made of different combustible materials in a series of ratios. Initial E. coli concentration was ca. 1.5×10⁵/ml.

FIG. 30. Schematic diagram of a full-size ceramic pot filter.

FIG. 31. Comparison of flow rate and bacterial LRV among ceramic filters in the present work and previous studies.

FIG. 32. Cumulative flow rate percentage as a function of pore size distribution of ceramic filters made of different combustible materials.

FIG. 33. The relationship between measured LRV and predicated LRV of the ceramic filters in the present work based on the semi-quantitative model.

FIG. 34. As(III) and As(V) removal through packed columns using granular La-coated ceramic materials. The contact time is ˜10 seconds. The initial concentrations of As(III) and As(V) were both 50 ppb.

FIG. 35. As(III) and As(V) removal through packed columns using La-coated ceramic disk. The contact time is ˜40 seconds. The initial concentrations of As(III) and As(V) were both 50 ppb. Practically 100% of the As was removed when the water was flowing through the disk.

FIG. 36. Bacteriophage MS2 (ATCC-15597-B1) removal at different pH.

FIG. 37. Effects of ionic strength and NOM concentration of bacteriophage MS2 log reduction value (LRV).

FIG. 38. La-modified and unmodified ceramic filter removal of MS2. Virus (MS2) initial concentration: ˜1.5×10⁴ PFU/mL.

FIG. 39. Comparison of raw clay compositions.

### DETAILED DESCRIPTION

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### 2. Filter Materials, Device, and Use

The filter material of the present invention may generally be made of a ceramic clay and may be used uncoated or may have a lanthanum-containing coating. The filter material may have various shapes depending on the desired application and it may be incorporated into filtration devices for use in removing contaminants from a water supply.

The uncoated ceramic clay has a network of pores that have a shape and a volume defined by combustion of cellulose fibers in a mixture of the cellulose fibers and a raw clay material. The pores acquire the shape and volume imparted by the dimensions and the distribution of cellulose fibers in the raw clay material upon combustion.

The mixture of cellulose fibers and raw clay material may comprise from 5-40% by weight of the cellulose fibers. Depending on the desired application, the mixture may comprise 5-35%, 5-20%, 5-15%, 5-10%, 10-35%, 10-20%, 10-15%, 10-12%, 15-35%, 15-25%, 15-20%, 20-35%, 20-30%, 20-25%, 8-12%, 13-17%, 18-22%, 23-27%, 28-32%, 10%, 15%, 20%, or 30% by weight of the cellulose fibers, or “about” the foregoing weight percentages of the cellulose fibers.

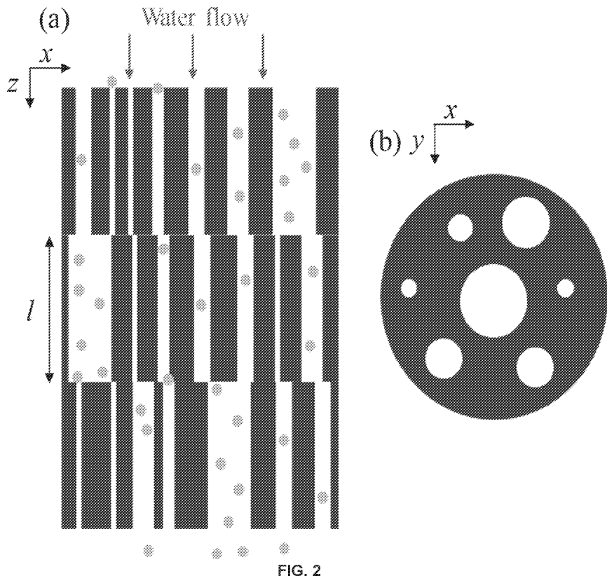

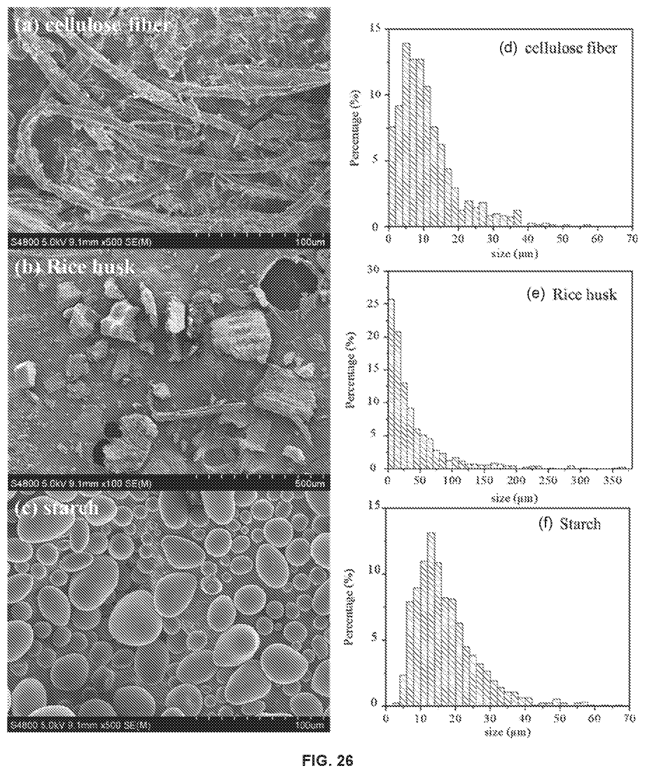

The cellulose fibers have a generally tubular shape as shown in FIG. 26A. Thus, the pore network comprises a series of tubular interconnected pores, as generally illustrated in FIG. 2. Preferably, the cellulose fibers are paper fibers, such as recycled paper fibers. The fibers may range in diameter from about 0.1 μm to 100 μm, with a median fiber diameter of 1-10 μm. The size distribution of the fibers may be further subdivided, wherein about 50-60% of the cellulose fibers have a diameter between 0 and 1 μm, about 30-40% of the cellulose fibers have a diameter from 1 to 10 μm, and about 5-10% of the cellulose fibers have a diameter from 10 to 100 μm. The cellulose fibers may have the size distribution shown in FIG. 26D.

The architecture and dimensions of the pores may vary with the amount of cellulose fiber used in the mixture with the raw clay material. Depending on the weight % of cellulose fibers, the pores may have a size distribution wherein 10-60% of the pores have a diameter from >0 to 1 μm, 20-60% of the pores have a diameter from 1 to 10 μm, and 5-50% of the pores have a diameter from 10 to 100 μm. Poor sizes may be measured by Mercury Intrusion Porosimetry (MIP).

The pores may have a size distribution wherein 50-60% of the pores have a diameter from >0 to 1 μm, 30-40% of the pores have a diameter from 1 to 10 μm, and 5-10% of the pores have a diameter from 10 to 100 μm. The pores may have a size distribution wherein 60-90%, 65-85%, 70-80%, 74-76%, or about 75% of the pores have a diameter <2 μm. For example, the pore size distribution for 10 wt. % fiber may fall within these ranges.

The pores may have a size distribution wherein 30-40% of the pores have a diameter from >0 to 1 μm, 50-60% of the pores have a diameter from 1 to 10 μm, and 10-20% of the pores have a diameter from 10 to 100 μm. The pores may have a size distribution wherein 37-67%, 42-62%, 47-57%, 50-54%, or about 52% of the pores have a diameter <2 μm. For example, the pore size distribution for 15 wt. % fiber may fall within these ranges.

The pores may have a size distribution wherein 20-30% of the pores have a diameter from >0 to 1 μm, 50-60% of the pores have a diameter from 1 to 10 μm, and 20-30% of the pores have a diameter from 10 to 100 μm. The pores may have a size distribution wherein 30-60%, 35-55%, 40-50%, 43-47%, or about 45% of the pores have a diameter <2 μm. For example, the pore size distribution for 20 wt. % fiber may fall within these ranges.

The pores may have a size distribution wherein 10-20% of the pores have a diameter from >0 to 1 μm, 35-45% of the pores have a diameter from 1 to 10 μm, and 40-50% of the pores have a diameter from 10 to 100 μm. The pores may have a size distribution wherein 12-42%, 17-37%, 22-32%, 25-29%, or about 27% of the pores have a diameter <2 μm. For example, the pore size distribution for 30 wt. % fiber may fall within these ranges.

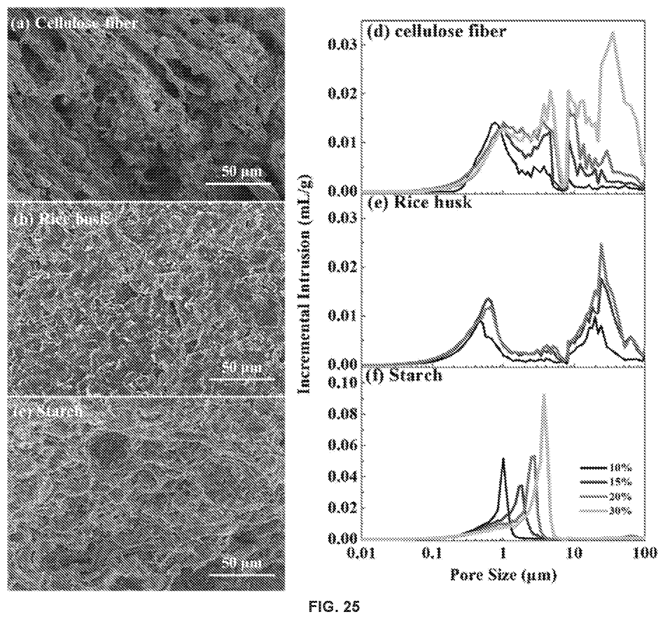

The pores may have the size distribution as generally shown in FIG. 25D. The pores formed in the ceramic clay from combustion of cellulose fibers may have an average pore size of 0.9-9 μm.

The ceramic clay has a void volume, a total bulk volume, a porosity, and permeability. The void volume comprises the volume occupied by the plurality of pores contained within the ceramic clay, while the total bulk volume is the volume of the ceramic clay. In other words, the void volume is the volume of pore (that is, void) space contained within the total bulk volume of the ceramic clay. The ratio of the void volume to total bulk volume is the porosity of the ceramic clay. The porosity of the ceramic clay is the fraction of the ceramic clay volume capable of being occupied by a fluid. The void volume comprises one or both of interconnected and non-interconnected void volumes (that is, interconnected and non-interconnected pores). The porosity of the ceramic clay also varies with the amount of cellulose fiber used in the mixture with the raw clay material. Depending on the weight % of cellulose fibers, the ceramic clay may have a porosity of 10-50%, 10-40%, 10-30%, 10-20%, 10-15%, 40-50%, 30-50%, 20-50%, 15-45%, 15-35%, 15-25%, 15-20%, 13-17%, 20-24%, 22-26%, 37-41%, about 15%, about 22%, about 24%, or about 39%.

The interconnected pores have a total interconnected pore surface area. In one embodiment, the total interconnected pore surface area of the ceramic clay is at least about 0.5 m² per gram of the uncoated ceramic clay. In a preferred embodiment, the total interconnected pore surface area of the ceramic clay is at least about 1 m²/g. In a more preferred embodiment, the total interconnected pore surface area of the ceramic clay is at least about 5 m²/g.

The permeability of the ceramic clay is a measure of the ease, with which a fluid will flow through the interconnected void volumes (that is, interconnected pores). A high porosity ceramic clay having substantially most, if not all, of the void volumes interconnected will have a substantially higher permeability than another ceramic clay having the same porosity but substantially most, if not all, of the void volumes non-interconnected.

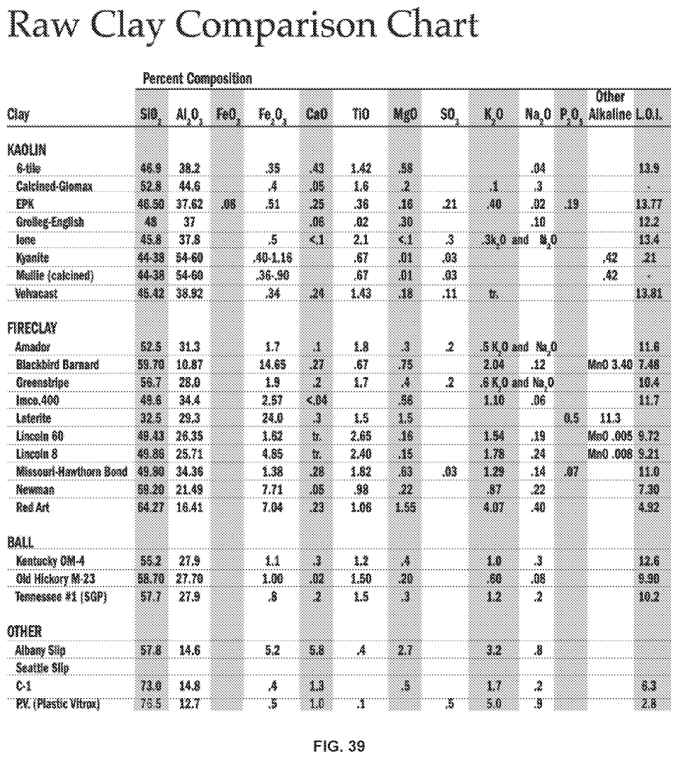

The ceramic clay may be a ceramic of a fireclay, a kaolinite, an illite, a zeolite, diatomaceous earth, or a montmorillonite. Fireclays include Amador, blackbird/barnard, Greenstripe, Imco 400, laterite, Lincoln 60, Lincoln 8, Missouri-Hawthorn blend, Newman, and red art. Kaolinites include 6-tile, Calcined-Glomax, EPK, Grolleg-English, lone, Kyanite, calcined Mullie, Velvacast, dickite, and nacrite. The ceramic clay may be obtained from the raw clay of any of those listed in FIG. 39. In a preferred embodiment, the raw clay is red art clay or blackbird clay.

The raw clay material may comprise 30-90 wt. % SiO₂ and 2-40 wt. % Al₂O₃. The raw clay material may comprise 30-70 wt. % SiO₂, 10-40 wt. % Al₂O₃, and 1-30 wt. % Fe₂O₃. The raw clay material may comprise 58-68 wt. % SiO₂, 10-20 wt. % Al₂O₃, and 5-15 wt. % Fe₂O₃. The raw clay material may comprise 62-66 wt. % SiO₂, 14-18 wt. % Al₂O₃, and 12-16 wt. % Fe₂O₃. The raw clay material may comprise 58-62 wt. % SiO₂, 9-13 wt. % Al₂O₃, and 5-15 wt. % Fe₂O₃.

The ceramic clay may be fashioned into various shapes during manufacture. For example, the ceramic clay may be formed as granules, a powder, a disk, a column, or a pot. In one aspect, the clay has a mesh size (i.e., relating to the number of holes per linear inch of a sieve screen) of about 30 mesh (about 595 μm). In another aspect, the clay has a mesh size of 10, 20, 40, 50, or up to about 100 mesh or some range therebetween (about 2000, 841, 400, 297 or down to about 149 μm). In alternate embodiments, the mesh size is about 20 mesh (about 840 μm). Granules correspond to a mesh size of 10 to 110 mesh. In some embodiments, the granules correspond to a mesh size of 18 to 45 mesh.

The ceramic clay may be prepared by forming a mixture of the raw clay material, cellulose fibers, and water. The weight percent of the cellulose fibers may be as described herein. Water is added to the mixture in an amount convenient to allow homogenization. Following homogenization of the mixture of raw clay, cellulose fibers, and water, the mixture may be molded into a desired shape conducive to efficient firing. These forms may take the form of pot, cup, tube, cylinder, disk, box, candle, bucket, in-line filter, or any other suitable form. The homogenized, molded mixture may be dried for an appropriate period of time before firing. A typical drying time is about two days at ambient temperature. The dried mixture is then fired by subjecting the mixture to incremental increases in temperature up to around 900-1000° C. Kilns and firing technology are well known to those of skill in the art and are well described in literature such as The Kiln Book, Materials, Specifications and Construction, by Frederick Olsen (Chilton Book Co., second edition, 1983), which is incorporated by reference herein.

Generally speaking, firing begins slowly at a preliminary firing temperature, especially through the ignition point, typically between about 500 to 600° C. After the cellulose fiber has burned off, the firing may be allowed to proceed at a rapid pace to a temperature higher than the preliminary firing temperature, up to 800° C. or greater, to the maturation temperature of earthenware, about 1000 to 1050° C. Lower or higher maximum temperatures (e.g., about 600, 700, 800, 900, 950, 1100, or 1150° C.) during firing are possible depending on the specific clay used and water content of the mixture being fired (e.g., clay mixtures with higher moisture or less coarse clay will use a slower temperature ramp). These values are easily determinable by those skilled in the art.

Firing continues until the firing mixture matures into earthenware and/or until the ceramic clay can no longer be broken down by water. Maturing temperatures and times typically depend upon the properties of the specific kiln, pottery oven, or firing device used. However, such properties are usually easily ascertainable by a user and determining the maturing temperature and time particular to a specific firing device does not require undue experimentation by one skilled in the art. Generally, a sufficient temperature not to be broken down by water is at least about 500° C., the firing mixture will be fired for at least about three hours. More preferably, the firing is at a temperature of about 600° C. Even more preferably, the firing is at a temperature of about 700° C. Most preferably, the firing is at a temperature of about 900° C. to about 1100° C., depending on the clay properties and moisture content. Generally, in a preferred embodiment, the firing will last for at least about three hours to about 24 hours, depending in large part on the size of the kiln.

For example, the firing temperature may be increased from ambient temperature to around 80-100° C., holding at this temperature range for about an hour, followed by further increasing the temperature to the maximal temperature and holding for an hour. In a representative protocol, the temperature is increased from ambient temperature at a rate of 60° C./hour to about 80° C., holding at about 80° C., increasing the temperature at a rate of 150° C./hour to 900° C. and holding at 900° C. for 1 hour. In another representative protocol, the temperature is increased from ambient temperature increasing the temperature at a rate of 14° C./hour to about 93° C., holding for 1 hour, then increasing at a rate of 83° C./hour to about 315° C., holding for 1 hour, increasing at a rate of 83° C./hour to about 593° C., holding for 30 minutes, and increasing at a rate of 70° C./hour to a final temperature of 1000° C. and holding for 1 hour. The material obtained after firing and cooling may be ground into granules or powders, as described herein.

Ceramic clay materials as described herein may have a lanthanum-containing coating. In some embodiments, the lanthanum-containing coating is a lanthanum (MII)-containing coating. For example, in some embodiments, the coating comprises LaONO₃, La₂O₃, LaOOH, La₂(CO₃)₃, and/or La₂O₂CO₃, depending on the coating protocol. The lanthanum coating is disposed on the outer surface of the ceramic clay and within the network of pores of the ceramic clay. In some embodiments, the lanthanum coating covers substantially all of the outer surface and the inner surfaces in the network of pores of the ceramic clay. In some embodiments, the coated ceramic clay has 0.5-25 wt. % of lanthanum (e.g., 18-22%). The lanthanum coating is sufficiently thin so as not to substantially impair the permeability of the ceramic clay. The lanthanum coating forms an insoluble film within the interconnected pores of the pore network. The lanthanum coating has an average film thickness. Preferably, the average film thickness is from about 0.5 nm to about 500 nm. More preferably, the average film thickness is from about 2 nm to about 50 nm. Even more preferably, the average film thickness is from about 3 nm to about 20 nm.

The lanthanum coating contained within interconnected pores has a total film surface area. In one embodiment, the total film surface area is at least about 0.5 m² per gram of the ceramic clay. In a preferred embodiment, the total film surface area is at least about 1 m² per gram of the ceramic clay. In a more preferred embodiment, the total film surface area is at least about 5 m² per gram of the ceramic clay. In some embodiments, the coated ceramic clay may display a Brunauer-Emmett-Teller (BET) surface area of 1-5.5 m²·g⁻¹, such as 5-5.5 m²·g⁻¹.

The lanthanum-coated ceramic clay of the invention is capable of adsorption of arsenic and chromium species, such as As(V), As(III), and Cr(VI). The lanthanum-coated ceramic clay may have an adsorption capacity for As(V) of 20-90 mg/g. In some embodiments, the adsorption capacity for As(V) may be 80-90 mg/g, 75-85, 70-80 mg/g, 65-75 mg/g, 60-70 mg/g, 55-65 mg/g, 50-60 mg/g, 45-55 mg/g, 40-50 mg/g, 35-45 mg/g, 30-40 mg/g, 25-35 mg/g, or 20-30 mg/g. The lanthanum-coated ceramic clay may have an adsorption capacity for As(III) of 2-25 mg/g. In some embodiments, the adsorption capacity for As(III) may be 20-25 mg/g, 15-20 mg/g, 10-15 mg/g, 5-10 mg/g, or 2-5 mg/g. The lanthanum-coated ceramic clay may have an adsorption capacity for Cr(VI) of 10-15 mg/g. In some embodiments, the adsorption capacity for Cr(VI) may be 15 mg/g, 14 mg/g, 13 mg/g, 12 mg/g, 11 mg/g, or 10 mg/g.

Adsorption capacity may vary with surface area, which in turn corresponds to pore size distribution, and ultimately, the weight % of cellulose fiber used to create the network of pores. Adsorption capacity may also be affected by access of the lanthanum compound to the network of pores during the coating process. For example, entrapped gases may prevent the coating material from completely penetrating the pore network, particularly for smaller diameter pores. For ceramic clays with a higher proportion of smaller pores, subjecting the ceramic clay to vacuum prior to coating can improve the adsorption capacity of the lanthanum-coated ceramic clay. Thus, for ceramic clay from 30 wt. % cellulose fiber, the adsorption capacity for As(V) may be 55-75 mg/g either with or without the vacuum treatment. For ceramic clay from 30 wt. % cellulose fiber, the adsorption capacity for As(III) may be 5-10 mg/g without vacuum treatment and 12-17 mg/g with vacuum treatment. For ceramic clay from 10 wt. % cellulose fiber, the adsorption capacity for As(V) may be 30-50 mg/g without vacuum treatment and 75-90 mg/g with vacuum treatment. For ceramic clay from 10 wt. % cellulose fiber, the adsorption capacity for As(III) may be 2-5 mg/g without vacuum treatment and 17-25 mg/g with vacuum treatment. For ceramic clay from 10 wt. % cellulose fiber, the adsorption capacity for Cr (VI) may be about 13 mg/g.

The lanthanum-coated ceramic clay may be prepared by treating the uncoated ceramic clay with one or more lanthanum salts selected from the group consisting of La(NO₃)₃, LaCl₃, LaBr₃, LaIN, LaF₃, La₂(SO₄)₃, LaPO₄, La₂(C₂O₄)₃, La₂O₃, LaOOH, La(OH)₃, La₂S₃, La(CH₃CO₂)₃, and LaAlO₃. The lanthanum salt used in the process may be in a mixture such as a solution, suspension, or dispersion in a fluid. Suitable fluids include, for example, water and/or organic solvents such as an alcohol (e.g., ethanol). Preferably, a lanthanum-containing solution comprises the lanthanum salt in a substantially dissolved state. It can be appreciated that the concentration of the lanthanum-containing solution is sufficiently concentrated to substantially coat at least some of the pores and pore volume of the ceramic clay. Moreover on the other hand, the concentration of the lanthanum-containing solution is sufficiently dilute so that the coating formed does not substantially diminish the permeability of the coated ceramic clay. In the lanthanum treatment step, the ceramic clay is contacted with the lanthanum-containing solution to form a wet-coated, impregnated ceramic clay. In one embodiment the ceramic clay is submerged into the lanthanum-containing solution, the submersion can be with or without agitation. The immersion time can be from about 0.1 hour to about 48, preferably from about 1 hour to about 24 hours. In yet another embodiment, the contacting of lanthanum-containing solution with the ceramic clay is by spray coating, curtain coating, dipping (completely or partially), kiss-coating, and coating under greater than atmospheric pressures. It can be appreciated, that, other coating methods well known within the art are likewise suitable.

Following the lanthanum treatment, the obtained material may be heated to from 100° C. to 800° C. In some embodiments, the heat treatment temperature may be from about 370° C. to 800° C. In some embodiments, the heat treatment temperature may be about 300° C. In some embodiments, the heat treatment temperature may be 370° C. to 400° C., including about 385° C. In some embodiments, the heat treatment temperature may be 480° C. to 520° C., including about 500° C. In some embodiments, the heat treatment temperature may be about 800° C. Following heating the coated ceramic clay may be cooled, rinsed (e.g., with water), and dried.

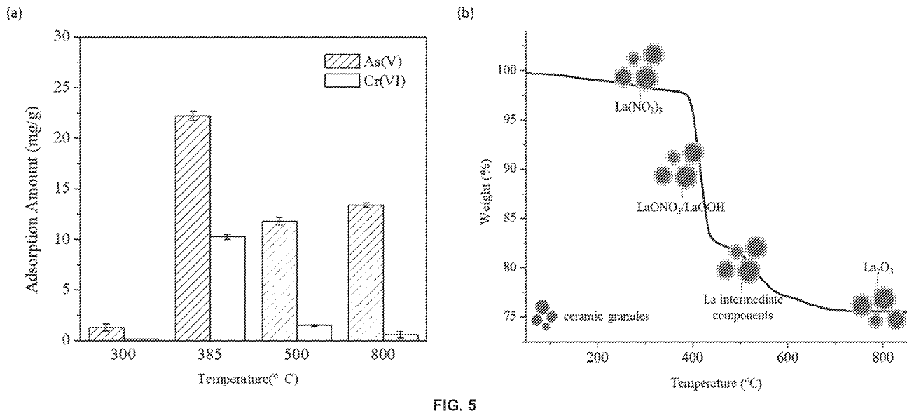

The lanthanum species present in the coating may vary with heat treatment temperature, as shown in FIG. 5B. Thus, in the range of about 300° C., the coating may comprise La(NO₃)₃. In the range of about 385° C., the coating may comprise LaONO₃ and/or LaOOH. In the range of 500° C., the coating may comprise La₂O₂CO₃. In the range of about 800° C., the coating may comprise La₂O₃.

In some embodiments, the coated ceramic clay may display FTIR peaks at about 3554, 1450, and/or 1300 cm⁻¹.

The coated and uncoated ceramic clay materials may be incorporated into filter devices for removal of contaminants from a water supply. The housing may have opposing first and second ends, an inlet, an outlet, and an outer wall extending between the first and second ends enclosing a fluid flow path between the inlet and the outlet. The housing may be in the form of a column, cartridge, or other like device and may be made of any suitable materials, such as metals, plastics such as PVC or acrylic, or other insoluble materials that will maintain a desired shape under conditions of use.

Filtration devices containing the coated or uncoated ceramic clays may be used to remove a contaminant from a water supply by contacting the water supply with the filtration device. At least one of the one or more contaminants is one of a chemical contaminant, biological contaminant, microbe, microorganism, virus and a mixture thereof. Generally uncoated ceramic clays may be used to remove bacterial or viral contaminants, whereas coated ceramic clays may be used to remove As(V), As(III), and/or Cr(VI).

In one embodiment, the disclosed apparatus and process effectively removes arsenic from fluids containing particularly high concentrations of contaminants. Arsenic concentrations in the fluid may be about 50 ppb to about 150 ppb. The disclosed apparatus and process are effective in decreasing the contaminants to levels safe for human exposure to the fluid (such as, for human consumption and/or inhalation of the fluid). For example, when the fluid contains arsenic the disclosed apparatus and process effectively decreases the arsenic level to amounts less than about 20 ppb, in some cases less than about 10 ppb, in others less than about 5 ppb, in still others less than about 2 ppb, and in still others substantially all arsenic.

In another embodiment, the disclosed apparatus and process effectively removes biological contaminants from fluids containing particularly high concentrations of the biological contaminants. The apparatus and process can effectively decrease one or more biological contaminants contained within the contaminant-containing fluid by from about 1 Log 10 to about 10 Log 10, more preferably from about 3 Log 10 to about 7 Log 10. Even more preferably from about 4 Log 10 to about 6 Log 10.

It can be appreciated, the level to which the one or more contaminants are decreased in the fluid can depend on one or more of: i) the initial contaminant level in the fluid, ii) the contaminant (as for example, without limitation, the chemical and/or physical properties of the contaminant); iii) the conditions under which the contaminant and apparatus are contacted (as for example, without limitation, one or more of contacting temperature and/or length of contacting time); iv) the apparatus physical properties (such as, without limitation, the apparatus size, permeability, and/or pore structure); and v) combinations thereof.

The porosity and permeability can affect the contacting pressure needed to achieve flow fluid through the filter device. The contaminant-containing fluid can flow through the filter device under the influence of gravity, pressure or other means and with or without agitation or mixing. While not wanting to be limited by any theory, the contacting pressure for the contaminant-containing fluid to flow through the filter device decreases the greater one or both of porosity and permeability of the filter material.

The contaminant-containing fluid is in contact with the filter material for a period of time. Preferably, the contact time can be as little as about 10 seconds. In another embodiment, the contact time can be about 40 seconds. In another embodiment, the contact time can be 1 minute or less. In another embodiment, the contact time can be about 1 to about 20 minutes. In yet another embodiment, the contact time can be from about 0.5 hours to about 12 hours. The contact time can vary depending on one or more of the geometry and size of the filter material, the porosity and/or permeability of the filter material, the contacting pressure, the fluid properties (such as viscosity, surface tension) and the contaminant and contaminant concentration within the contaminant-containing fluid. The disclosed filter device can effectively remove one or more contaminants from the contaminant-containing fluid.

When the contaminant-containing fluid comprises a liquid fluid the filter device can remove the one or more contaminants over a wide range of pH levels. In some embodiments, the pH is about 4-11. In some embodiments, the pH is about 4-10. In some embodiments, the pH is about 4-7. In some embodiments, the pH is about 5-8.

### 3. Examples

### Example 1

### Materials and Methods

Materials. La(NO₃)₃.6H₂O, Na₂HAsO₄.7H₂O, HCl, NaOH, NaCl, NaNO₃, Na₂SO₄ and NaHCO₃, Na₂HPO₄, NaH₂PO₄.H₂O and Na₂SiO₃.5H₂O were in analytical grade and purchased from Fisher Scientific (USA). As(V) stock solution (1000 mg/L) was prepared by dissolving Na₂HAsO₄.7H₂a in water, and As(III) stock solution (7500 mg/L) was directly purchased from Ricca Chemical Company (USA). Both As(V) and As(III) working solutions were freshly prepared by diluting the corresponding stock solutions with water. Ultrapure water (resistivity >18.0 MΩ) was used for all experiments.

Preparation of ceramic granules. The ceramic materials used in this research were made of red art clay and cellulose fiber. The red art clay was obtained from Resco products Inc (USA). According to the manufacturer, the chemical composition was 64.2% SiO₂, 16.4% Al₂O₃, 7% Fe₂O₃, 4.1% K₂O, 1.6% MgO (wt %). Cellulose fiber, which was made from recycled materials such as used paper, was obtained from Greenfiber Inc. (USA). Both red art clay and cellulose fiber were used as received.

The red art clay was mixed with cellulose fiber and water in the ratio of 9:1:5 (by weight). The homogenized mixture paste was molded into small cylindrical pieces using plastic pipes. The clay cylinders were air-dried at room temperature for 2 days and then fired in an electronic kiln (Olympic Kilns, USA). The temperature configuration for the kiln firing was: 1) increase at a rate of 60° C./hour from room temperature to 80° C., holding for 3 hour; and 2) increase at a rate of 150° C./hour to 900° C., holding for 1 hour. After being taken out of the kiln, the ceramic cylinders were broken into smaller blocks and sieved for the fraction of 18 to 45 mesh sizes. The sieved ceramic grains were cleaned repeatedly through deionized (DI) water rinsing, dried at 105° C., and stored in plastic containers for characterization and further modification.

Modification of ceramic granules and disks by lanthanum nitrate. To prepare pristine ceramic materials, redart clay and cellulose fiber in a desired ratio were mixed with water. The paste was then molded into a cylindrical plaster, compressed by hand, and dried in air. To get an adequate material strength and flow rate of the resulting filters, the ratios of clay and cellulose fiber were 9:1 and 4:1 for ceramic granule and disk, respectively. The dried clay cylinders were fired in an electronic kiln at a final temperature at 900° C. for an hour. After cooling down, the fired ceramic cylinders for granules filters were grounded, sieved (18-45 mesh), rinsed with water, dried at 105° C. and then used in the following coating process. The fired ceramic cylinders for disk filters were shaped into a size of 6.5-cm in diameter and 1.4-cm in thickness before the coating process.

La was coated onto the ceramic surface using a wet impregnation method. Briefly, a 1.14×10⁶ mg/L (3.5 M) La(NO₃)₃ solution was added to immerse both ceramic granules and disks with a liquid-to-solid ratio of 0.5 mL/g (i.e., 0.5 mL of La(NO₃)₃ solution per 1 g of ceramic material), followed by heating at 385° C. for 3 h. After cooling down to room temperature, the La-coated ceramics were rinsed by water to remove the loosely attached La components, and then dried at 105° C. before use.

La-coated ceramic granules were prepared by fabrication of ceramic granules and La-coating on ceramic surface. A commercial lanthanum nitrate salt, La(NO₃)₃.6H₂O (Fisher Scientific), was used as the precursor for the surface modification of the ceramic granules. It is well established that, under the presence of ambient air, the thermal decomposition of La(NO₃)₃.6H₂O proceeds through a series of steps that include dehydration, decomposition to LaONO₃, La intermediate compounds and La₂O₃(Strydom et al., Thermochim. Acta 1988, 124, 277-283; Gobichon et al., Solid State Ion. 1996, 93, 51-64; Mentus et al., J. Therm. Anal. Calorim. 2007, 90, 393-397). The thermal treatment of La(NO₃)₃.6H₂O-amended ceramic materials thus could allow for the systematic investigation on the effects of different types of La(III) modification on the removal of As and Cr(VI).

Granular ceramic sorbent prepared above were firstly immersed in a saturated La(NO₃)₃ solution at a 1:1 (mass:volume) ratio. The mixtures were then heated for 3 hour in a furnace (Thermo Scientific, USA) at 300° C., 385° C., 500° C. and 800° C., respectively. The treated ceramic granules were then cooled at room temperature and repeatedly rinsed with DI water to remove any remaining free-forms of La. The modified ceramic granules were then dried in oven at 105° C. and stored in polypropylene bags before further use.

Batch adsorption experiments for As(V) and Cr(VI). Batch experiments were conducted at room temperature (22±2° C.) to determine the adsorption of As(V) and Cr(VI) by selected La-modified granular ceramic sorbents without pH adjustment. The pH was stable at ˜6.8 over the course of the experiments. The As(V) and Cr(VI) stock solutions (1000 mg/L) used in this research were prepared from Na₂HAsO₄.7H₂O and Na₂CrO₄.4H₂O (Fisher Scientific, USA), respectively. To determine the adsorption kinetics, ceramic granules were mixed with As(V) (20 mg/L) or Cr(VI) (3 mg/L) solutions in centrifuge tubes that were mixed on a rotator (Techne TSB3, USA). The ceramic granule loadings were 1.0 g/L and 0.5 g/L for As(V) and Cr(VI), respectively. At preselected times after the initiation of the adsorption experiments (e.g., 1 min, 5 min up to 48 hours), 3 tubes were randomly selected, and the liquid in each tube was filtered using 0.22-μm cellulose acetate filters (VWR International, USA). As(V) concentrations in the filtrates were measured by inductively coupled plasma atomic emission spectroscopy (ICP-AES, Perkin Elemer Optima 2100 DV, USA) and Cr(VI) concentrations were determined by the 1,5-diphenylcarbazide method (Federation, American Public Health Association (APHA): Washington, D.C., USA, 2005). The quantify of adsorbed As(V) or Cr(VI), qₑ (mg/g), was calculated by the following mass balance equation:

$${q}_{e} = \frac{( {C}_{i} - {C}_{e} ) V}{W} \qquad ( 1 )$$

where Cᵢ and Cₑ (mg/L) are the initial and final or equilibrium arsenic or Cr(VI) concentration in solution, respectively. V (mL) is the volume of arsenic or Cr(VI) solution, and W (g) is the mass of ceramic materials. Results from the adsorption kinetics experiments indicated that adsorption equilibrium was generally reached within 24 hours.

The As(V) and Cr(VI) adsorption isotherms were subsequently obtained through similar batch experiments. Ceramic granules were mixed with As(V) or Cr(VI) solutions in 50-mL centrifugal tubes at a ratio of 1.0 g:1000 mL (for As(V)) or 1.0 g:2000 mL (for Cr(VI)). The initial As(V) and Cr(VI) concentrations varied between 6 mg/L and 75 mg/L and between 1.5 mg/L and 70 mg/L, respectively. Following 24 hours of mixing, the solution in each tube was filtered through 0.22-μm cellulose acetate filter and the concentrations of As(V) and Cr(VI) in the filtrates were quantified as previously described. The quantities of adsorbed As(V) and Cr(VI) were determined using equation (1).

Batch adsorption experiments for As(I) and As(V). Batch adsorption experiments were performed using La-coated ceramic granules to determine the adsorption behavior of As(V) and As(III). Experiments were conducted by mixing La-coated ceramic granules with 292.2 mg/L (5 mM) NaCl solutions (mass/volume ratio=1/1000) containing either As(III) or As(V) at pH 6.0 in 50 mL centrifugal tubes on a rotator (Techne TSB3, USA) at room temperature (22±2° C.), unless otherwise specified. High arsenic concentrations were used to allow for full evaluation of the performance and capacity of the La-coated ceramic materials. Specifically, adsorption kinetics were determined by collecting samples in suspensions with 5 mg/L of As(V) or As(III) at a series of predetermined time intervals (10 min to 24 h). As(III) and As(V) adsorption isotherms were determined by varying their initial concentrations (4-50 mg/L for As(III) and 10-100 mg/L for As(V)); samples were collected after 24 hours of reaction. Additionally, sorption of As(V) and As(III) was investigated as a function of initial solution pH (i.e., 4-11) and various coexisting anions under environmentally relevant concentrations (i.e., chloride, 0-177 mg/L (0-5 mM); nitrate, 0-310 mg/L (0-5 mM); sulfate, 0-480 mg/L (0-5 mM); bicarbonate, 0-305 mg/L (0-5 mM); silicate, 0-76.1 mg/L (0-1 mM); phosphate, 0-9.60 mg/L (0-0.1 mM)) (Drever, Prentice Hall Englewood Cliffs: New Jersey, 1988, Chapter 9, 167-206; Faust et al., Ann Arbor Science: Michigan, USA, 1981, Chapter 1, 3-24; Walther, Second ed. Jones & Bartlett Learning: Sudbury, Mass., USA, 2009, 259). A higher As(V) concentration (20 mg/L) was used in these experiments than As(III) (5 mg/L), because of their different sorption affinities. Solution pH was adjusted using 3650 mg/L (0.1 M) HCl or/and 4000 mg/L (0.1 M) NaOH solutions.

In all experiments, once collected, samples were immediately filtered using 0.22-μm syringe filters (cellulose acetate), acidified to 2% HNO₃ and preserved for analysis. Arsenic concentrations were quantified by ICP-AES. The quantity of adsorbed arsenic, qₑ (mg/g), was calculated by Equation (1). Where C, and Ce (mg/L) are the initial and equilibrium arsenic concentration, respectively. V (L) is the volume of arsenic solution, and W (g) is the mass of ceramic materials.

Material characterization. The physicochemical and morphological properties of unmodified and La-coated ceramic materials were analyzed before and after arsenic sorption. Scanning electron microscopy (SEM, Hitachi S-4800 FE-SEM, Japan) imaging was carried out to characterize the morphology of ceramic granules before and after La-modification. The La content in the unmodified and La-modified ceramic granules was quantified by extracting the materials using 2% HNO₃, filtering the extraction liquid using 0.22-μm cellulose acetate filters, and measuring La concentration in the filtrates by inductively coupled plasma atomic emission spectroscopy (ICP-AES, Perkin Elemer Optima 2100 DV, USA). Surface properties of La-coated ceramic granules were analyzed before and after arsenic adsorption. X-ray diffraction (XRD) patterns were obtained by Bruker D8 discover X-ray Diffractometer using Cu Kα radiation, with a scan rate (20) of 4°·min−1 (λ=1.5418 Å). The specific surface area was measured via nitrogen adsorption through the Brunauer-Emmett-Teller (BET) method using a Micromeritics ASAP 2000 surface area analyzer (Micromeritics Co., USA). Fourier transform infrared spectroscopy (FTIR) analysis was performed to determine the material surface functionality by using a Bruker Vector 22 spectrometer (Bruker, Germany). The vibrations corresponding to the wavenumbers ranging from 650 to 4000 cm⁻¹ were collected with the resolution of 4 cm⁻¹. X-ray photoelectron spectroscopy (XPS) was applied to investigate the coordination environment of arsenic using a Perkin Elemer pHi 5440 ESCA system with Mg Kα radiation. The binding energies obtained in the XPS analysis were calibrated against the C is peak at 284.8 eV. Zeta potential values were measured by a Zetasizer Nano ZS90 (Malvern Instruments, UK). Zeta potentials of samples at a series of pH were measured and the point of zero change (pH_{PZC}) of sample was derived by linear interpolation of the two points above and below the x-axis. Thermogravimetric analysis (TGA) was performed on a TA SDTQ650 (TA instruments, US) in the temperature range of 50 to 850° C. with an air flow rate of 100 mL/min and a heating rate of 10° C./min. The La concentrations in both the raw and La-coated ceramic materials were determined by acid digestion (2% HNO₃), followed by measurement of the filtered digestate (0.22 μm cellulose acetate syringe filters) using inductively coupled plasma atomic emission spectroscopy (ICP-AES, Perkin Elemer Optima 2100 DV, USA).

Ceramic filter fabrication and characterization. Redart clay, cellulose fiber, rice husk, and potato starch were the main ingredients used for the ceramic filter fabrication. Redart clay was obtained from Resco products Inc. (USA). Cellulose fiber, a processed recycle paper fiber, was purchased from Greenfiber Inc. (USA). Both rice husk and potato starch were purchased from local market. The redart clay, cellulose fiber and potato starch were used as received, and the rice husk was sieved by 18-mesh sieves (<1 mm) before use (Van der Laan et al., Water Res. 2014, 51, 47-54).

The morphology of cellulose fiber, rice husk and potato starch were investigated by SEM (Hitachi S4800 FE-SEM, Japan) imaging and their size distributions were obtained by image analysis. At least 500 measurements of each sample were taken for size distribution analysis.

Ceramic porous media were fabricated in the shape of disk to simplify the geometry in the lab-scale studies (relative to pot and tubular shapes) (Oyanedel-Craver at al., Environ. Sci. Technol. 2008, 42, 927-933; Rayner et al., ACS Sustain. Chem. Eng. 2013, 1, 737-745). Briefly, the redart clay was mixed with an individual combustible material (i.e., Cellulose fiber, rice husk, or potato starch) at dry weight ratios of 90%:10%, 85%:15%, 80%:20%, and 70%:30%, respectively. Deionized (DI) water was then added to the dry mixture of clay and combustible materials, and a mixer (KitchenAid, USA) was used to homogenize the wet mixture. 160 g wet mixture of each recipe was then transferred into a cylindrical plaster mold and compressed for 1 min by hand. The resulting greenwares were air-dried at room temperature for at least 2 days before firing. To get an adequate material strength from all recipes, ceramic disk filters were fired at a final temperature of 1000° C. based on a firing protocol modified from our previous study (Yang et al., CS Sustain. Chem. Eng. 2019, 7, 9220-9227). For each mixing ratio, at least three disk filters were fabricated. Because all filters fabricated using 30% rice husk and 70% redart clay showed cracks after kiln firing, this specific ratio was not further tested in this study.

The porosity and pore size distribution of the ceramic disk filters were measured using mercury intrusion porosimetry (MIP, Micromeritics AutoPore IV, USA). SEM imaging was carried out to determine the morphology of ceramic disk filters. The elemental composition of all filters was measured on a Bruker S4 PIONEER XRF using the method described by McHenry (McHenry, Chem. Geol. 2009, 265, 540-552). FTIR analysis was performed using a Bruker Vector 22 spectrometer (Bruker, Germany) to determine the functional groups of the filters. The vibrations corresponding to the wavenumbers ranging from 650 to 4000 cm⁻¹ were collected with a resolution of 4 cm⁻¹.

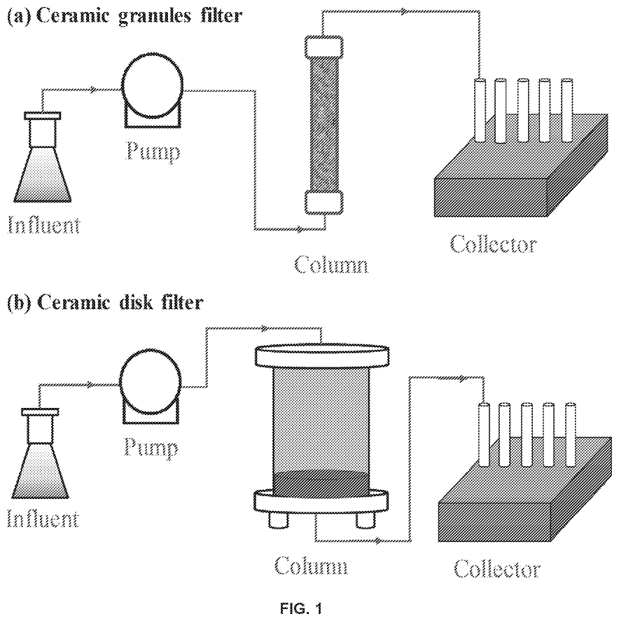

Filtration experiments. Two types of bench-scale prototype filters, namely ceramic granules-packed column filter and ceramic disk filter, were developed to quantify the performance of La-coated ceramic materials for As(III) and As(V) removal (FIG. 1). Ceramic granules filter was prepared by wet packing of La-coated ceramic granules in a glass column with an inner diameter of 1.0 cm and a length of 15 cm. The porosity of the packed ceramic granule columns were ˜46%. A Masterflex peristaltic pump (Cole-Parmer, USA) was used to introduce an arsenic-containing (As(III) or As(V)) solution from an influent reservoir to the granules filter with a flow rate of 1 mL/min. The performance of ceramic disk filter was evaluated by clamping a ceramic disk (diameter of 6.5-7.5 cm, thickness of 1.4 cm) on the bottom of a custom-built acrylic column with an inner diameter of 6.5-7.5 cm and a length of 11.0 cm. Prior to each filtration experiment, ceramic disk was wetted and sterilized by boiling in DI water for at least 1 hour. After the wetted ceramic disk was cooled, it was placed at the bottom of the column and sealed to the column wall by a water-tight sealant. Sterilized DI water was pumped through the ceramic disk filter till the flow rate was constant. A flow rate of 2 mL/min was maintained with the use of a Masterflex pump. Influents for both prototype filters were prepared by spiking As(V) or As(III) into a 292.2 mg/L (5 mM) NaCl solution at pH 6.0 to a concentration of 0.120 mg/L (As(V)) or 0.125 mg/L (As(III)). This baseline condition was selected because of its relevance to natural aquatic systems contaminated with arsenic (Sorg et al., Water Res. 2014, 48, 156-169; Drever, Prentice Hall Englewood Cliffs: New Jersey, 1988, Chapter 9, 167-206: Atekwana et al., Hydrol. Process. 2004, 18, 2801-2815; Faust et al., Ann Arbor Science: Michigan, USA, 1981, Chapter 1, 3-24; Jiang et al., Int. J. Environ. Heal. R. 2013, 10, 18-46). Each experiment was run for more than two months. Effluents were collected using a fraction collector and measured by ICP-MS (Thermo Scientific Element 2, USA) for arsenic concentration quantification.

For filtration of bacteria-containing fluid, 1 L E. coli spiked DI water with a concentration of 1.5×10⁵ CFU/mL was allowed to pass through an uncoated ceramic filter disk by gravity to investigate the filtration performance of the clean filter in its early lifespan. In practice, clean ceramic filters are generally tested in the factories or lab to evaluate their performance before application or further pretreatment (e.g. impregnation with silver) (Oyanedel-Craver at al., Environ. Sci. Technol. 2008, 42, 927-933; Rayner et al., J Water Sanit. Hyg. De. 2013, 3, 252-261). During the time course of the filtration experiment, a constant water level was maintained within the column using a peristaltic tubing pump (Masterflex, USA). Flow rate of the effluent was quantified by measuring the weight of effluent samples per desired duration time. Effluent samples were collected at selected time intervals and the E. coli concentrations were immediately quantified via the plate counting method. These data had been used to determine the average flow rate and comparison of E. coli concentration between influent and effluent (Van der Laan et al., Water Res. 2014, 51, 47-54; Bielefeldt et al., Water Res 2009, 43, 3559-3565).

For filtration experiments, bacterial log reduction values (LRVs) were calculated based on Equation (2) to quantify bacterial removal efficiency by the filters:

LRV=log₁₀(Cᵢₙ)−log₁₀(C_{eff}) (2)

where Cᵢₙ and C_{eff} (CFU/mL) are the E. coli concentrations in the influent and effluent, respectively. The detection limit of ˜6 LRV (˜99.9999% removal) was determined by using a maximum sample volume of 10 mL for plate counting.

Semi-quantitative microscale model for bacterial removal efficacy. The interconnected pores within the ceramic filters provide flow pathways for water. The pore network within the ceramic filters is similar to that of natural porous media such as soil and sand. The parallel bunch of capillaries model was widely used to simplify the geometry of porous media (Bedrikovetsky, Transport Porous Med. 2008, 75, 335-369; You et al., SPE J. 2013, 18, 620-633). It is assumed that similar to packed natural sands, a porous ceramic disk filter can be divided into many layers in the direction of flow and each layer consists of a parallel bunch of capillaries (FIG. 2). Within each layer the size of capillaries follows the measured overall filter pore size distribution and remains intact during low-retention filtration (Scheidegger, University of Toronto Press 1974). The water flow (Q) through each microscopic capillary of size x is described by the Hagen-Poiseuille law (Equation 3):

$$Q = \frac{π {x}^{4}}{32 μ} \frac{Δ P}{Δ L} \qquad ( 3 )$$

where μ is the viscosity of water and

$$\frac{Δ P}{Δ L}$$

is pressure gradient across each layer. Given the same viscosity and pressure gradient, the flow rate is proportional to x⁴, which means that the water flow has a strong preference for large pores.

Given any probability distribution function p(x) of pore size x for the ceramic filter, the fractional water flow through pore size between x₁ to x₂ of a single layer can be calculated based on the following equation (integration of Equation (3)):

$$\frac{{\int }_{{x}_{1}}^{{x}_{2}} Q ( x ) p ( x ) d x}{{\int }_{0}^{∞} Q ( x ) p ( x ) d x} = \frac{{\int }_{{x}_{1}}^{{x}_{2}} \frac{π {x}^{4} Δ P}{32 μΔ L} p ( x ) d x}{{\int }_{0}^{∞} \frac{π {x}^{4} Δ P}{32 μΔ L} p ( x ) d x} = \frac{{\int }_{{x}_{1}}^{{x}_{2}} {x}^{4} p ( x ) d x}{{\int }_{0}^{∞} {x}^{4} p ( x ) d x} \qquad ( 4 )$$

Assuming straining as the only mechanism of bacteria capture and that the attachment removal is negligible, bacterial cells can be immobilized within pores that are smaller than the cell size. As a result, the probabilities that bacteria pellets are captured (Pₛ) and passed through a layer (Pₚ) are expressed as Equation (5) and (6), respectively:

$$\begin{matrix}P s = \frac{{\int }_{0}^{{x}_{cell}} {x}^{4} p ( x ) d x}{{\int }_{0}^{∞} {x}^{4} p ( x ) d x} & ( 5 ) \\ {P}_{p} = 1 - {P}_{s} & ( 6 )\end{matrix}$$

where x_{cell} is bacteria pellet size which is considered 2 μm for E. coli in this case (Xu et al., Environ. Sci. Technol. 2008, 42, 771-778). Consequently, the probability that E. coli cells can pass through the entire ceramic disk filter is shown in Equation (7):

P=(1 −Pₛ)_{L/l} (7)

where L is the thickness of ceramic disk filter and I is the thickness of each microscopic layer as a reference length parameter of porous media that has the same order of magnitude of a media pore length (e.g., the thickness of a layer of sand in a sand pack) (Bedrikovetsky, Transport Porous Med. 2008, 75, 335-369; You et al., SPE J. 2013, 18, 620-633; Chalk et al., Chem. Eng. Res. Des. 2012, 90, 63-77). The overall removal efficiency of E. coli cells (Kr) is thus calculated by Equation (8):

Kᵣ=1−P=1−(1 −Pₛ)^{L/l} (8)

Adsorption experiments using natural water sources. To further investigate the adsorption performance of La-modified ceramic material, batch and column experiments may be conducted at several types of natural water sources, including (1) model solutions as baseline (pH=7, pollutant only), (2) Lake Michigan water, (3) a groundwater source, and (4) a representative wastewater. An individual pollutant may be added to the water sources to desired concentrations (e.g., 100 μg/L for As(III), As(V) and Se(VI), and ˜2×10⁴ PFU/mL for virus).

Long-time adsorption experiments. To evaluate the lifetime of La-modified ceramic material as adsorbent in point-of-use water treatment, column and disk filtration experiments may be conducted until breakthrough.

Column experiments: Dynamic flow adsorption experiments were conducted in a glass column with an inner diameter of 1.0 cm and a length of 15 cm. The bed volume (BV) of the column was 11.8 cm³. The column was packed with 12.0 g La-coated ceramic granules, resulting a porosity of 46%. Water sources mentioned above spiked 100 μg/IL As(III)/As(V) or ˜2×10¹ PFU/mL MS2 were used as influent solutions. The columns were operated in an upflow with a flow rate of 1.0 mL/min controlled by a Masterflex peristaltic pump (Cole-Parmer, USA). For As(III) column runs, nitrogen gas was used to prevent As(III) from oxidation during column experiment process. The effluent solution was collected at different time intervals, and the concentration of arsenic in the effluent solution was monitored by ICP-MS (Thermo Scientific Element 2, USA) after acidified by 2% HNO₃. The concentrations of the viruses will be determined following EPA Method 1602.

Disk filtration experiments: Filtration experiments were performed in an open-top custom-built acrylic column of inner diameter 6.5 and length 11 cm. Ceramic filter was clamped on the bottom of the column and sealed by putty (WM Harvey, USA) to make sure the water cannot leak from the edge of the column. The filter then was further saturated by going through at least 1 L sterilized DI water. Following saturation, water contained pollutant (As(III), As(V), E. coli, MS2) treated by the ceramic disk. During the whole filtration experiments, the column was connected to a Masterflex pump that maintained a flow rate to keep a constant water level (10.5 cm) in the column. Flow rate of effluent was monitor during the experiment. The effluent samples were collected as a function of time until the concentration of pollutant exceed EPA standards.

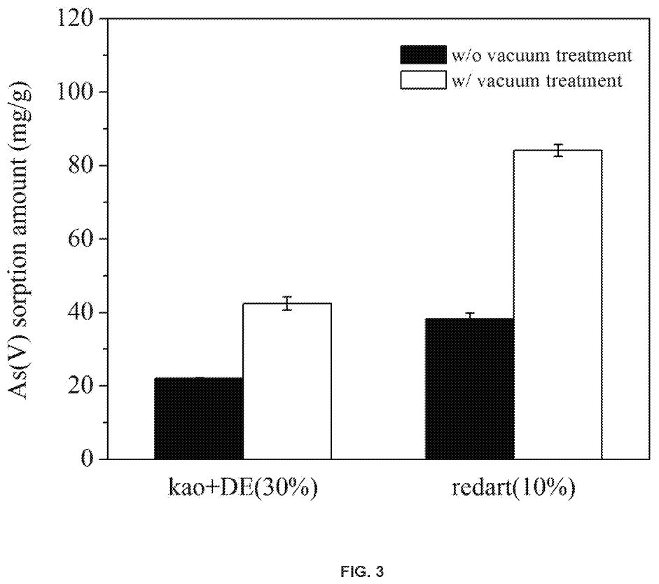

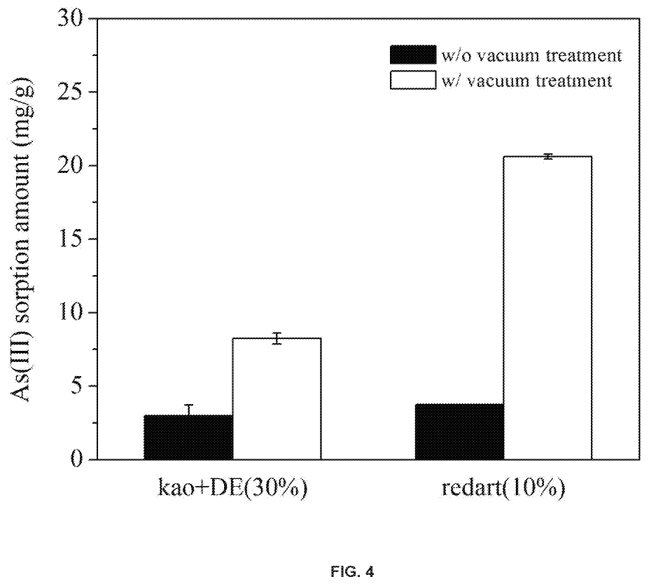

Preparation of La-modified materials using vacuum coating. A vacuum was used to remove air from pores, particularly from capillary pores, which may not be accessible to La coating solution due to trapped air, which has low solubility in water. Further the vacuum was used to remove CO₂ in air which can complicate the coating process through producing various amounts of La₂(CO₃)₃. Various ceramic materials, including ceramic materials made using redart clay, diatomaceous earth and/or kaolinite, and redart clay, were used. First, the materials were put into a vacuum flask. After the flask was sealed with a stopper, the vacuum was applied to remove air. Following the air-removal step, de-gassed La(III) solution was introduced into the flask to immerse the target materials (e.g., ceramic materials). The La(IU) modified materials were then thermally treated at ˜385° C. The La(UI) modified materials under vacuum and non-vacuum (i.e., ambient air) conditions were tested for their adsorption of As(V) and As(III) (FIG. 3-FIG. 4). The major findings from the tests is that vacuum significantly increased the adsorption capacity of La-coated ceramic materials for both As(V) and As(III). For As(V) adsorption, the low-cost La(III)-modified ceramic materials exhibited ˜80 mg/g adsorption capacity for As(V), and ˜20 mg/g adsorption capacity for As(III).

Virus filtration. Bacteriophage MS2 (ATCC-15597-B1) was used as the example virus for the virus filtration experiments. Escherichia coli (ATCC 15597) was inoculated in tryptic soy broth liquid media and then infected with MS2. The MS2 suspension was separated from bacteria cells by centrifugation at 4000 rpm for 20 min and then was purified by precipitation with 10% (w/v) PEG6000 and 0.5 M NaCl. The mixture was centrifuged at 10000 rpm for 60 min. The supernatant was discarded, and the MS2 pellets were resuspended in sterilized ultrapure water and filtered through a 0.45 μm cellulose acetate syringe filters. The residual broth was further washed by deionized water in a 100 kDa Amicon Ultra centrifugal filter unit (MilliporeSigma, USA) for two times. The final MS2 stock was stored in 10 mM PBS at 4° C. MS2 was enumerated by the double agar layer procedure as described in USEPA Method 1602. The MS2 stock solution was diluted to obtain the target concentration of ˜1.5×10⁴ PFU/mL for the filtration experiments.

Ceramic filter was placed in one end of a tubing and sealed by a hose clamp. A Masterflex pump (Cole-Parmer, USA) was employed to regulate the flow rate during all filtration experiments. MS2 virus (˜1.5×10⁴ PFU/mL) spiked in 5 mM NaCl solution was introduced to ceramic filter by pump. The effluent samples were collected at a series of time intervals (0.5-18 days). MS2 concentrations in effluents were immediately quantified via enumeration by double ager layer procedure. MS2 LRVs (log reduction values) were calculated based on Equation (9) to quantify removal efficiency by the filters:

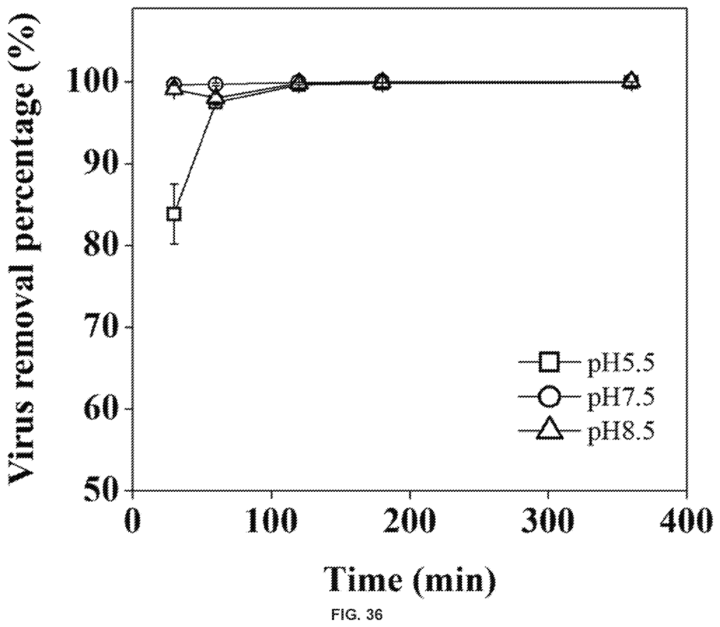

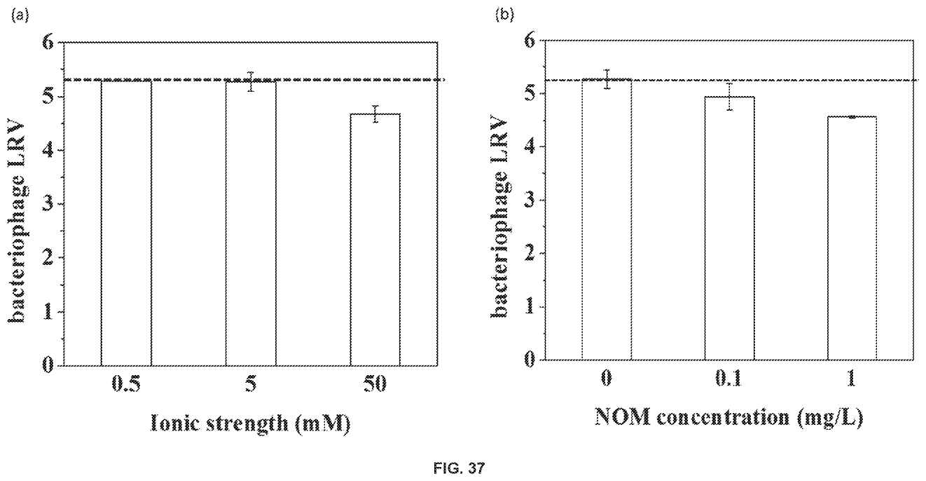

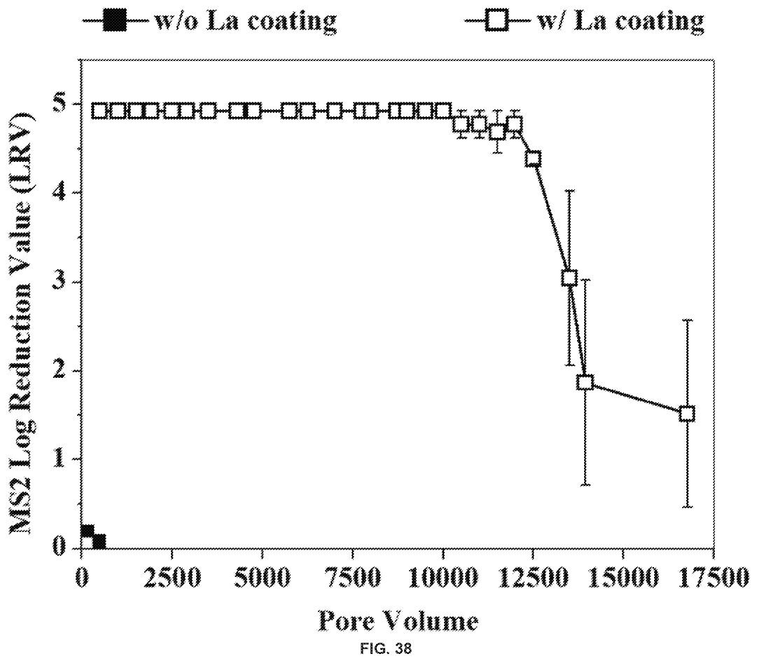

LRV=log₁₀(Cᵢₙ)−log₁₀(C_{eff}) (9)

where Cᵢₙ and C_{eff} (PFU/mL) are the MS2 concentrations in the influent and effluent, respectively. The detection limit of ˜5 LRV or 99.999% was determined by using a maximum sample volume of 5 ml for plate counting. See FIG. 36, FIG. 37, and FIG. 38 for virus removal results.

### Example 2

### As(V) and Cr(VI) Adsorption by Unmodified and La-Modified Ceramic Granules Treated at Different Temperatures

When La(NO₃)₃ was used as precursor, the temperature selected for the thermal treatment step could significantly impact both the composition and crystalline structure of La coating (Strydom et al., Thermochim. Acta 1988, 124, 277-283; Gobichon et al., Solid State Ion. 1996, 93, 51-64; Mentus et al., J. Therm. Anal. Calorim. 2007, 90, 393-397). In this research, the La(NO₃)₃ and ceramic material mixtures were thermally treated at 300° C., 385° C. and 800° C. to represent dehydration, formation of LaONO₃, and formation of La₂O₃, respectively. A temperature of 500° C. was also selected to represent the formation of intermediate products during the transformation from LaONO₃ to La₂O₃. Single-point adsorption experiments were then performed to determine the background adsorption of As(V) and Cr(VI) by the unmodified ceramic adsorbent, as well as the effects of temperature selected for the thermal treatment step on As(V) and Cr(VI) adsorption by the La-modified ceramic sorbent.

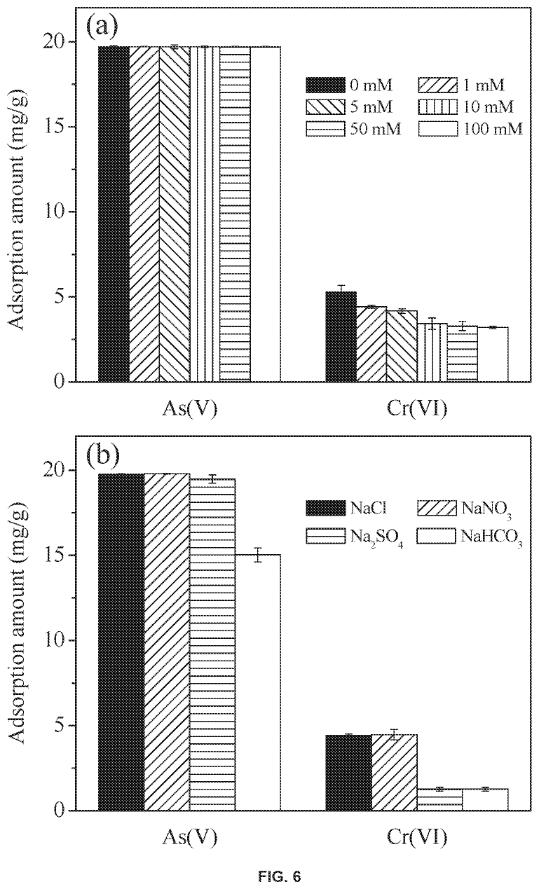

Our results showed that the unmodified ceramic granules had negligible adsorption for both As(V) and Cr(VI). For the ceramic granules treated at 300° C., As(V) adsorption was ˜1.5 mg/g and the adsorption of Cr(VI) was negligible (FIG. 5). The amount of adsorbed As(V) and Cr(VI) reached maximum values (22.2±0.4 mg/g for As(V) and 10.3±0.2 mg/g Cr(VI), respectively) at 385° C. Further increase in thermal treatment temperature to 500° C. and 800° C. resulted in lower As(V) and Cr(VI) adsorption. Since As(V) and Cr(VI) adsorption by the unmodified ceramic granules was negligible, our results showed that 1) La-modification was primarily responsible for As(V) and Cr(VI) adsorption; and 2) 385° C. was the optimal thermal treatment temperature for the La modification step. In the following section, detailed batch experiments were performed using La-modified ceramic granules that were treated at 385° C. FIG. 6 shows the effect of ionic strength in NaCl background solution (FIG. 6A) and coexisting anions (1 mM) on As(V) and Cr(VI) adsorption on La-modified ceramic granules (FIG. 6B). Initial As(V) concentration and adsorbent dosage were 20 mg/L and 1.0 g/L, and initial Cr(VI) concentration and adsorbent dosage are 3 mg/L and 0.5 g/L, solution pH is 6.0 adjusted by 0.1 mol/L HCl. Ionic strength had little influence on As(V) adsorption. In contrast, Cr(VI) adsorption was more sensitive to ionic strength than As(V) that increasing the ionic strength gradually decreased the adsorption of Cr(VI). The presence of Cl⁻, NO₃⁻, and SO₄⁻ had negligible effects on the removal of As(V), while the adsorption amount of As(V) decreased by ˜25% in the presence of HCO₃⁻. For Cr(VI) adsorption, Cl⁻ and NO₃⁻ also posed minimal effects on Cr(VI) removal, but strong inhibition was observed in the presence of SO₄²⁻ and HCO₃⁻ on Cr(VI) adsorption. The ceramic granules were also characterized to gain insights into its adsorption of As(V) and Cr(VI).

### Example 3

### Adsorption Kinetics and Isotherms of As(V) and Cr(VI)

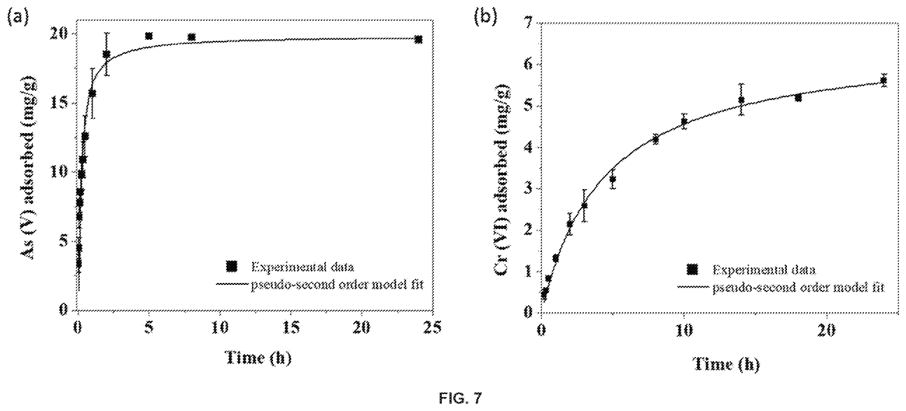

FIG. 7 shows the adsorption kinetics of As(V) and Cr(VI) by the La-modified ceramic granules treated at 385° C. Both kinetics curves exhibited a rapid initial uptake and the adsorption plateaued within ˜24 hours. The adsorption kinetics of As(V) observed in this research was faster than previously reported results while the adsorption kinetics of Cr(VI) was comparable to Cr(VI) adsorption by some La-amended adsorbents (Cui et al., PLOS ONE 2016, 11, e0161780; Zhang et al., Chem. Eng. J. 2014, 251, 69-79).

Both pseudo-first order and pseudo-second order kinetic models were used to fit the As(V) and Cr(VI) adsorption kinetics data. The two models are expressed in Equations (10) and (11), respectively (Ho et al., Process Biochem. 1999, 34, 451-465).

$$\begin{matrix}q = {q}_{e} ( 1 - {e}^{- {k}_{1} t} ) & ( 10 ) \\ q = {( \frac{1}{{q}_{e}} + \frac{1}{{k}_{2} {q}_{e}^{2}} {t}^{- 1} )}^{- 1} & ( 11 )\end{matrix}$$

Where qₑ and q stand for the quantities of adsorbed contaminant (mg/g) at equilibrium and at time t (h), respectively, while k₁ (h−1) and k₂ (g·mg⁻¹·h⁻¹) represent the rate constants for pseudo-first order and pseudo-second order kinetic models, respectively. Comparison of correlation coefficients (r²) (TABLE 1) showed that both As(V) and Cr(VI) adsorption kinetics could be better described with the pseudo-second order kinetic model, which corresponded to a chemisorption process (Ho et al., Process Biochem. 1999, 34, 451-465).

TABLE 1
Kinetics curve fitting parameters for adsorption of As(V) and Cr(VI)
on La-modified granular ceramic adsorbents treated at 385° C.

| | pseudo-first order kinetic model | | | pseudo-second order kinetic model | | |
|---|---|---|---|---|---|---|
| anion | kₗ | qₑ^{b} | | k₂ | qₑ^{b} | |
| species | (h⁻¹) | (mg · g⁻¹) | r² | (g · mg⁻¹ · h⁻¹) | (mg · g⁻¹) | r² |
| As(V) | 3.18 ± 0.50 | 18.7 ± 0.8 | 0.902 | 0.233 ± 0.035 | 19.8 ± 0.63 | 0.963 |
| Cr(VI) | 0.182 ± 0.016 | 5.74 ± 0.15 | 0.981 | 0.0328 ± 0.003 | 6.68 ± 0.12 | 0.995 |

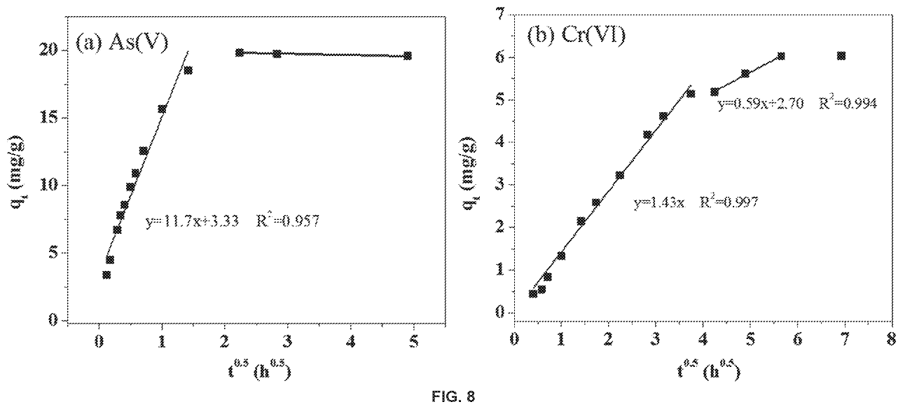

Additionally, to determine the possible role of intra-particle diffusion on anion adsorption process, the kinetic data was also fitted to Weber-Morris model (Weber et al., J. Sanit. Engng. Div. 1963, 89, 31-60). The linear form of Weber-Morris model was given in Equation (12):

$${q}_{t} = {k}_{i} {t}^{\frac{1}{2}} + C \qquad ( 12 )$$

Where qₜ was amount of adsorbed contaminant at time t, kᵢ was the intra-diffusion rate constant (mg·g⁻¹·h^{-0.5}), and C was the intercept which represents the thickness of the boundary layer. The multi-linearity of both Cr(VI) and As(V) curves indicates that intra-particle diffusion was not only the rate-controlling step for adsorption (FIG. 8). For Cr(VI), the two regions with different slopes suggest a boundary layer adsorption (Gupta et al., J. Hazard Mater. 2009, 163, 396-402) (initial steep phase), followed by a gradual sorption controlled by intra-particle diffusion (second less steep phase). In contrast, the intra-particle diffusion stage was not obvious for As(V) sorption. Results suggested that although the intra-particle diffusion was not necessarily the sole rate-determining step for As(V) or Cr(VI) adsorption, intra-particle diffusion had stronger influence on Cr(VI) adsorption than on As(V) adsorption.

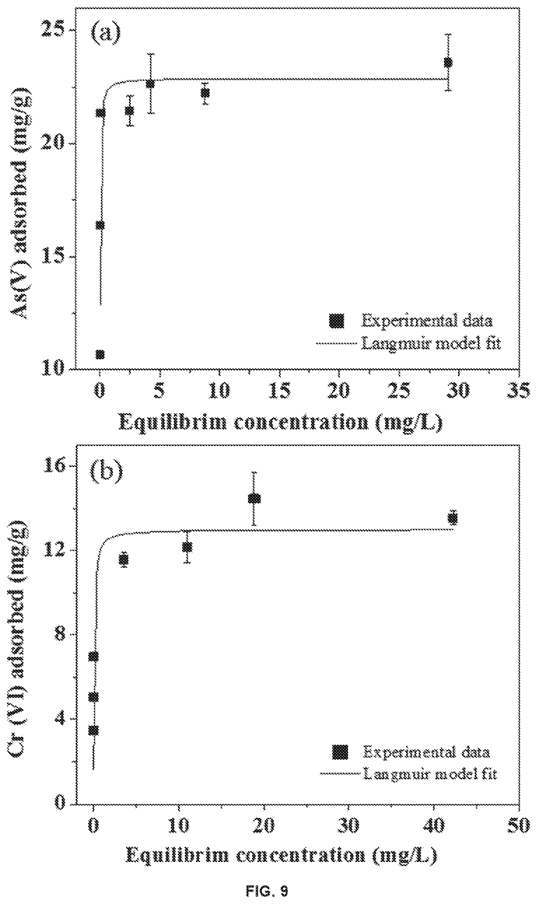

As(V) and Cr(VI) adsorption isotherms are presented in FIG. 9. For both chemical species, the adsorption by the La-modified ceramic materials increased sharply at low aqueous concentrations, suggesting the high affinity of the sorbents with As(V) and Cr(VI). The adsorption data were fitted to the Langmuir adsorption isotherm model (Langmuir, J. Am. Chem. Soc. 1918, 40, 1361-1403; Freundlich, Zeitschrift fur Physikalische Chemie 1906, 57, 385-470):

$${q}_{e} = \frac{{q}_{m} {K}_{L} {C}_{e}}{1 + {K}_{L} {C}_{e}} \qquad ( 12 )$$

Where Cₑ (mg/L) is equilibrium concentration of contaminants in solution, qₑ (mg/g) represents the amount of adsorbed contaminants per unit mass of adsorbent and K_{L} is the Langmuir affinity constant related to energy of adsorption (Guo et al., Environ. Sci. Technol. 2005, 39, 6808-6818).

The adsorption parameters derived by fitting the isotherm model are summarized in TABLE 2. The estimated adsorption capacity for As(V) and Cr(VI) by the La-modified ceramic granules was 22.9±0.9 mg/g and 13.0±0.6 mg/g, respectively. The As(V) adsorption capacity was comparable to a reported La-modified sawdust sorbent, and was 511% and 48% higher than La-impregnated silica gels and Fe-modified ceramic materials, respectively (TABLE 3) (Setyono et al., ACS Sustain. Chem. Eng. 2014, 2, 2722-2729; Chen et al., Colloid Surface A 2012, 414, 393-399; Chakravarty et al., Water Res. 2002, 36, 625-632; Wasay et al., Water Environ. Res. 1996, 68, 295-300). The Cr(VI) adsorption capacity was at least 56% higher than those of bituminous coal, biochar, CTAB modified silica gelatin and alkyl ammonium surfactant bentonite (Di Natale et al., J. Hazard Mater. 2007, 145, 381-390; Showkat et al., B. Kor. Chem. Soc. 2007, 28, 1985; Sarkar et al., J. Hazard Mater. 2010, 183, 87-97). The La-modified ceramic materials developed in this research can thus serve as a sustainable, low-cost and effective sorbent for the removal of As(V) and Cr(VI) from drinking water sources.

TABLE 2
Isotherm fitting parameters for adsorption of As(V) and Cr(VI)
on La-modified granular ceramic adsorbents treated at 385° C.

| anion | Langmuir model | | |
|---|---|---|---|
| species | qₘ(mg · g⁻¹) | K_{L}(L · mg⁻¹) | r² |
| As (V) | 22.9 ± 0.9 | 70.8 ± 18.5 | 0.839 |
| Cr (VI) | 13.0 ± 0 6 | 16.1 ± 4.7 | 0.913 |

TABLE 3
Comparison of adsorption of As(V)
and Cr(VI) on various adsorbents.

| | | solu- | adsorption | |
|---|---|---|---|---|
| | | tion | capacity | |
| Sorbates | Adsorbents | pH | (mg · g⁻¹) | reference |
| As(V) | La-modified | 7.0 | 28.6 | Setyono et al., |
| | sawdust | | | 2014 |
| | Fe-impregnated | 6.9 | 8.49 | Chen et al, |
| | ceramic | | | 2012 |
| | La-impregnated | 7.0 | 3.75 | Wasay et al., |
| | silica gel | | | 1996 |
| | manganese ore | 6.5 | 15.4 | Chakravarty et |
| | | | | al., 2002 |
| | La-modified | 6.8 | 22.9 | this study |
| | ceramic | | | |
| Cr(VI) | Alkyl ammonium | 5.0 | 8.36 | Sarkar et al., |
| | surfactant bentonite | | | 2010 |
| | CTAB modified | 5.8 | 5.8 | Showkat et al., |
| | silica gelatin | | | 2007 |
| | Bituminous coal | 5.0-8.0 | 7.0 | Di Natale et al., |
| | | | | 2007 |
| | La-modified | 6.8 | 13.0 | this study |
| | ceramic | | | |

### Example 4

### Characterization of La-Modified Granular Ceramic Adsorbents

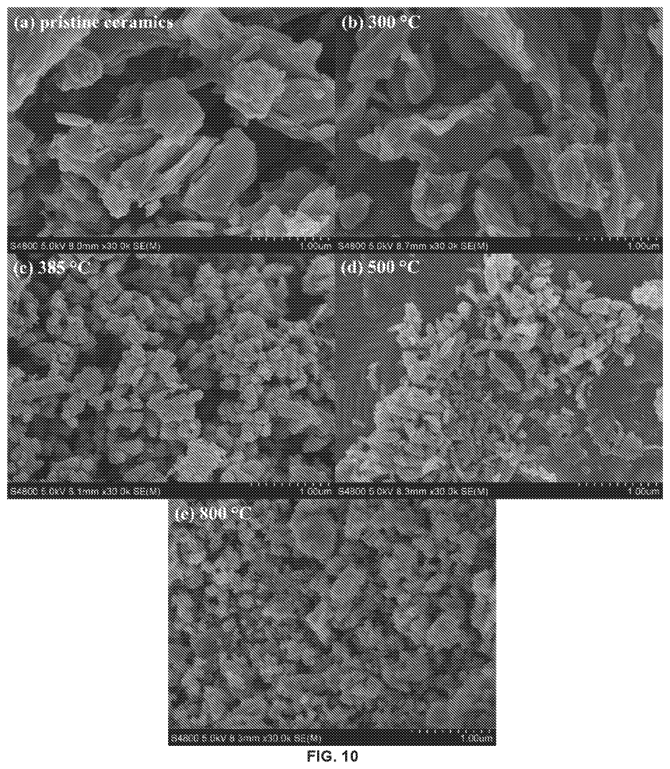

SEM images of the unmodified and La-modified ceramic adsorbents were obtained to examine their surface morphology (FIG. 10). The surface of the ceramic materials was dominated by micrometer scale plate-shaped structures. The surface of La-modified ceramic materials that were thermally treated at 300° C. appeared similar to the surface of the unmodified ceramic materials. The surface of La-modified ceramic granules that were thermally treated at 385, 500 and 800° C., however, was covered by high densities of fine particles, indicating the successful coating of La on ceramic surfaces.

The measurement of BET surface area also showed that the unmodified and 300° C. La-modified ceramic materials had similar surface areas (2.79 and 2.65 m²/g, respectively), both of which were significantly lower than the surface area of La-modified ceramic materials that were thermally treated at 385° C. and above (TABLE 4). This increase in BET surface area could be attributed to the fine La-containing particles coated on the surface of the ceramic granules.

TABLE 4
BET surface area and La content percentages of La-modified granular
ceramic materials treated at different temperatures.

| | La content percentage | BET surface area |
|---|---|---|
| Sample | (wt %) | (m² · g⁻¹) |
| w/o | ND | 2.79 |
| modification | | |
| 300° C. | 0.65 ± 0.01 | 2.65 |
| 385° C. | 20.4 ± 0.6 | 5.24 |
| 500° C. | 24.8 ± 0.4 | 4.47 |
| 800° C. | 25.8 ± 0.5 | 6.44 |

The quantity of La extracted from unmodified ceramic materials was below detection limit. TABLE 4 showed that for the La-modified ceramic materials that were thermally treated at 300° C., the fraction of La was less than 2% (w.t.) whereas amount of La increased sharply to 20.4% at 385° C. and remained constant at higher temperatures of 500 and 800° C. Results are consistent with the morphology and surface area measurements, showing that La modification was only stable at temperature ≥385° C.

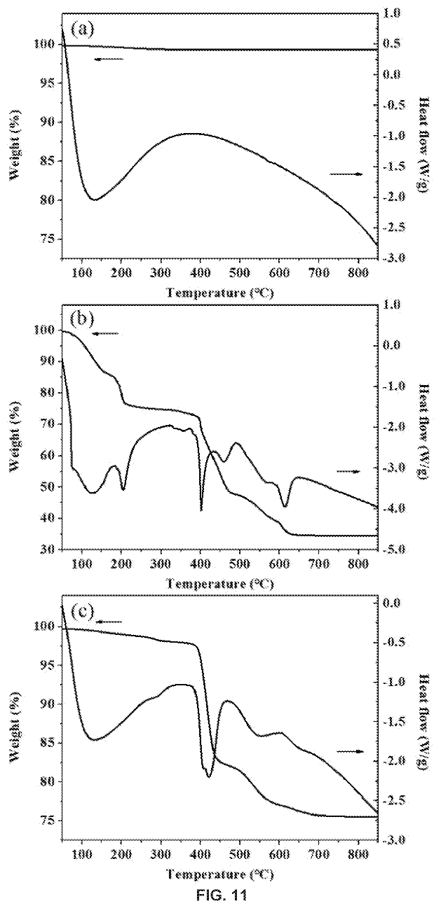

To further determine the composition and structure of La surface coating, TGA was performed for La(NO₃)₃.6H₂O, unmodified ceramic materials and La-modified ceramic materials. For the unmodified ceramic materials, negligible weight loss was observed during thermal heating process (FIG. 11C), indicating ceramic material was stable during thermal treatment as high as 800° C. As shown in FIG. 11A, there were four weight loss steps in the TGA curve of La(NO₃)₃.6H₂O that corresponded to dehydration, formation of LaONO₃, decomposition to intermediate components, as well as the conversion from intermediate components to La₂O₃, respectively. These weight loss steps and the corresponding temperatures were in excellent agreement with the dehydration and chemical transformation steps observed in previous studies (Gobichon et al., Solid State Ion. 1996, 93, 51-64; Mentus et al., Therm. Anal. Calorim. 2007, 90, 393-397). The TGA weight loss curve for the La-modified ceramic materials (FIG. 11B) was consistent to that of La(NO₃)₃.6H₂O alone (FIG. 11A). The TGA results indicated that the La compounds coated on the surface of the ceramic materials underwent similar thermal transformation as La(NO₃)₃.6H₂O.

For the La-modified ceramic materials that were treated at 300° C., the resulting La coating on the surface is likely La(NO₃)₃, which could be easily removed through repeated DI water rinsing due to its high water solubility. This could explain the minimal change of surface morphology, the low La content extracted using diluted HNO₃, and the lack of adsorption capacity for As(V) and Cr(VI) by the La-modified materials treated at 300° C.

When the La-modified ceramic materials were treated at 385° C., the resulting surface coating was predominantly LaONO₃ and related ligand exchange products in water (e.g., LaOOH). This form of coating is stable and displayed high affinity for the adsorptive removal of As(V) and Cr(VI). Thermal treatment at higher temperatures transformed LaONO₃ into intermediate La compounds such as La₂O₂CO₃ and finally to La₂O₃ (800° C.). These transformations did not lead to any measurable loss of La content, but significantly lowered the adsorption capacity for As(V) and Cr(VI).

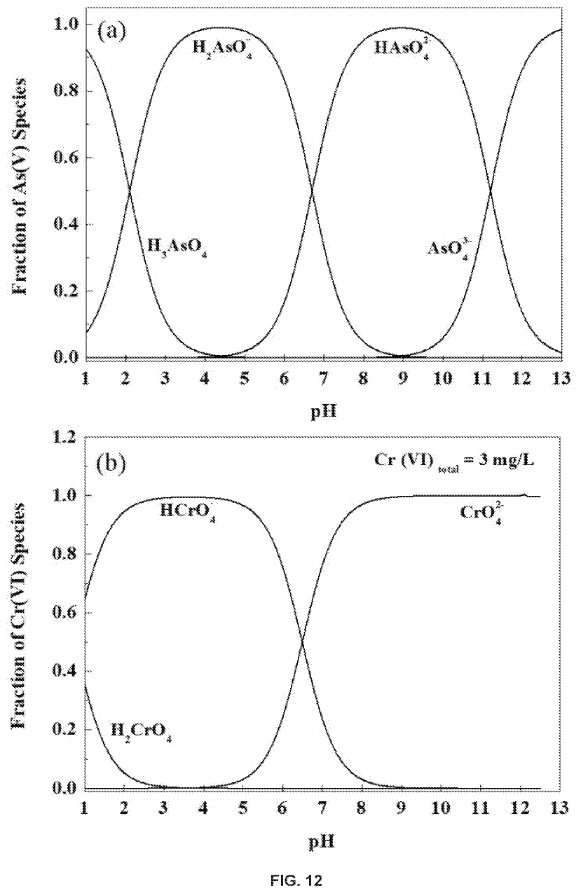

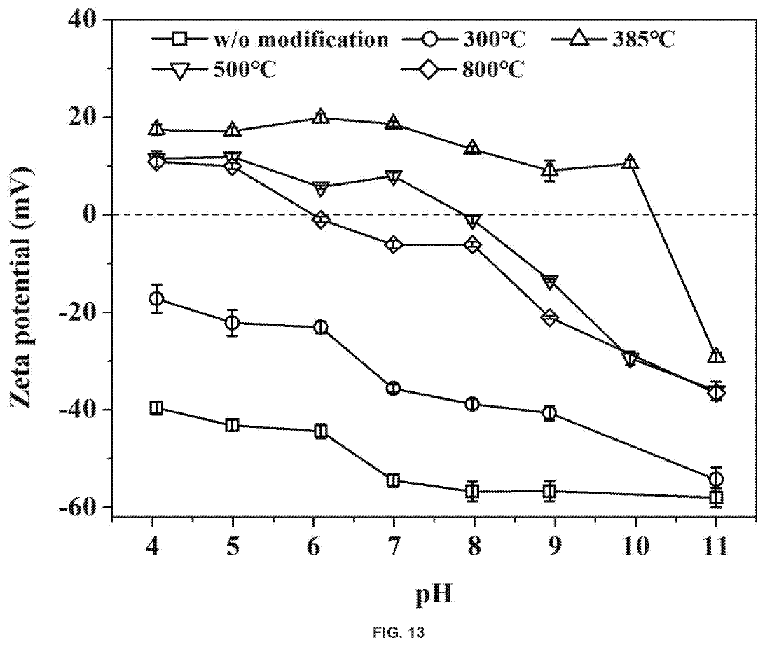

Surface charge of unmodified and La-modified ceramic sorbents could also be closely related to their adsorption behavior for As(V) and Cr(VI), the speciation of which changes dramatically with pH (FIG. 12). Zeta potential of unmodified and La-modified ceramic particles prepared at different thermal treatment temperatures were measured as a function of pH. For the unmodified ceramic adsorbent, the zeta potential was very negative (<−40 mV) under relatively acidic conditions. The zeta potential dropped by ˜20 mV as pH increased from 4 to 11 (FIG. 13). The negative charges on the surface of the unmodified ceramic materials would lead to a repulsive interaction between the negatively charged As(V) (e.g., H₂AsO₄⁻, HAsO₄²⁻) and Cr(VI) (e.g., HCrO₄⁻, CrO₄²⁻) ions and prevent their adsorption. This was consistent to the negligible adsorption of As(V) and Cr(VI) by the unmodified ceramic granules.

Compared to the unmodified ceramic sorbent, surface coating by La compounds led to less negative surface charge at all pH conditions as well as a higher point of zero charge (pHPZC) (FIG. 13). It is well known that La compounds tend to be positively charged even under basic conditions (Kosmulski, CRC Press: 2009). Particularly, the surface of La-modified ceramic adsorbent treated at 385° C. was positively charged within the pH range of 4-10. The positive charge was probably caused by the dissociation of NO₃⁻ from LaONO₃ and/or the protonation of the associated ligand exchange products (e.g., LaOOH). As the main species of As(V) and Cr(VI) ions under environmentally relevant pH conditions (5.5-8.5) were all negatively charged (FIG. 12), the positively charged sites created by La surface coating promoted the adsorption of both Cr(VI) and As(V) anions through electrostatic attraction (Liu et al., Environ. Sci. Technol. 2015, 49, 7726-7734; Gheju et al., J. Hazard Mater. 2016, 310, 270-277; Deng et al., J. Hazard Mater. 2010, 179, 1014-1021). It is interesting to note that the La-modified ceramic sorbents treated at temperatures higher than 385° C. displayed lower zeta potential values and this observation was consistent to their lower adsorption capacity for both As(V) and Cr(VI).

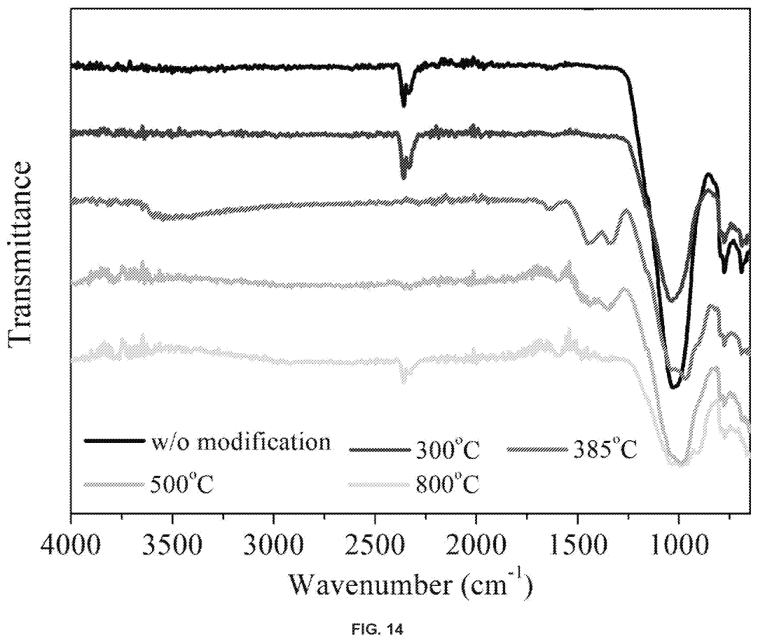

To further understand the functional groups on the ceramic surface, FTIR spectra for unmodified and La-modified ceramic granules at different firing temperatures were measured. As shown in FIG. 14, the band at 1030 cm⁻¹, ascribed to concerted (Si—O—Si) stretches (Jang et al., Micropor. Mesopor. Mat. 2004, 75, 159-168), was observed for ceramic granules both before and after La modification. Unlike the narrow and sharp peak for Si—O—Si stretch on the pristine ceramic surface, a broader and less intense peak around 1000 cm⁻¹ was obtained for all examined La-modified ceramic materials, which was assigned to the combination bands of La—O fundamental vibrational modes (Klingenerg et al., Chem. Mater. 1996, 8, 2755-2768) and Si—O—Si stretch. By comparing FTIR spectra for La-modified ceramic materials among different firing temperatures, significant new peaks at 3554, 1450 and 1300 cm⁻¹ were observed for La-modified sorbent at 385° C., which corresponded to O—H stretching group of La (hydr)oxide (Jais et al., Sep. Purif. Technol. 2016, 169, 93-102), stretching vibration of H—O—H and vibration mode of NO₃⁻, respectively. It is consistent with the observation of the main La compound (LaOOH/LaONO₃) at 385° C. from TGA profile. It is worth noting that these peaks were reduced in the sample modified at 500° C. and they disappeared in the sample modified at 800° C., indicating the change of surface functional groups during the high temperature treatment steps. Thus, the maximum As(V) and Cr(VI) adsorption by the La-modified ceramic sorbents treated at 385° C. could be related to the functional groups of LaOOH/LaONO₃ that are involved in the sorption process. Previous studies also reported that hydroxyl group induced by La modification played a dominant role in anions adsorption by La-modified red mud and alumina (Cui et al., PLOS ONE 2016, 11, e0161780; Shi et al., J. Mater. Chem. A 2013, 1, 12797-12803).

Based on the TGA, zeta potential, and FTIR analysis, increased adsorption of As(V) and Cr(VI) by the La-modified ceramic materials may be attributed to both enhanced electrostatic interaction and the formation of surface complexes between La surface functional groups and the anions. Different adsorption behaviors were observed between Cr(VI) and As(V) on La-modified ceramic materials, which was probably due to the distinct interaction between different anions and sorbents (Benjamin, Water chemistry. Waveland Press: 2014), and the exact mechanisms that govern the sorption of different anions worth further investigation.

### Example 5

### Characterization of La-Coated Ceramic Sorbents

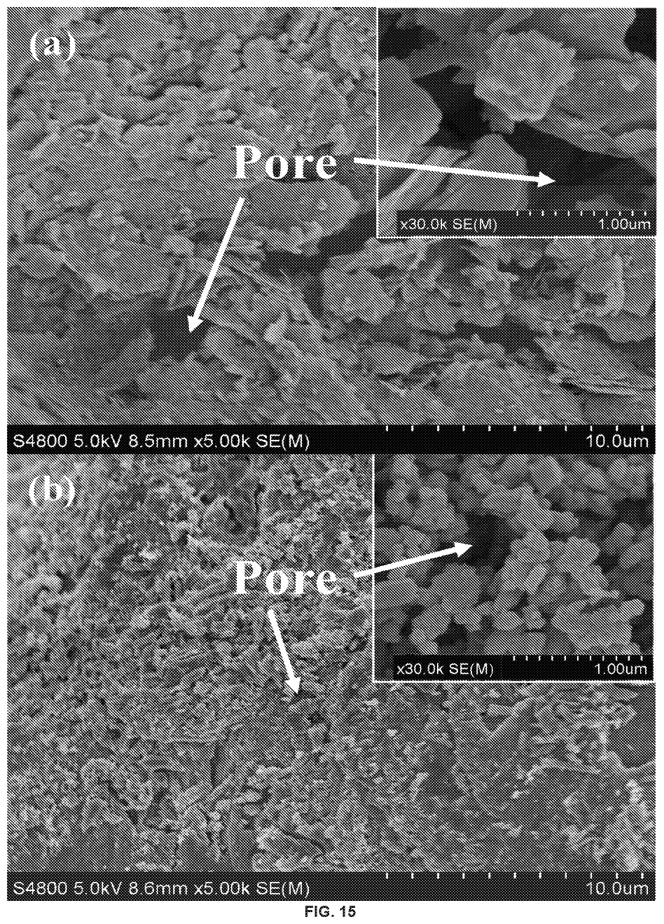

The morphological and physicochemical properties of the raw and La-coated ceramic materials were characterized with a variety of analytical techniques. SEM images showed that the uncoated ceramic material exhibited a porous structure with pore sizes ˜0.5-2 μm (FIG. 15A), while small particles were observed to cover the surface of La-coated ceramic material (FIG. 15B), suggesting that La coating influenced the morphology of ceramic surface. Higher BET surface area was found for the La-coated ceramic material (5.2 m²/g) than the uncoated material (2.8 m²/g), which was consistent with the change of the surface morphology after La coating. ICP-AES analysis of the acid digestates of the ceramic materials suggested that the La loading was 20.4 wt % for the La-coated ceramic material; in contrast, no La was found for the uncoated ceramic material.

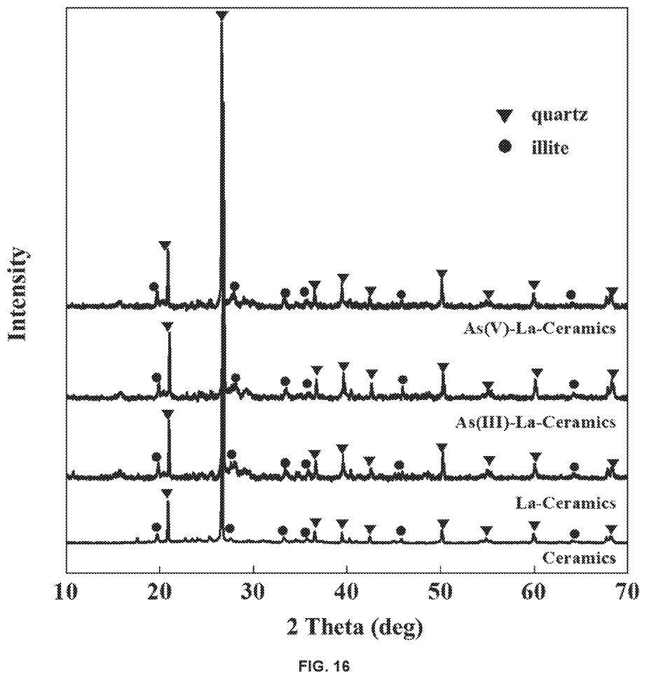

XRD patterns were obtained to further examine the crystalline phases of the raw and La-coated ceramic materials. Characteristic peaks of quartz (SiO₂, PDF #00-005-0490) and illite (K_{0.5}(Al,Fe,Mg)₃(Si,Al)₄O₁₀(OH)₂, PDF #00-009-0343) were clearly observed for the raw ceramic material (FIG. 16), suggesting that they were the predominant phases for the raw ceramic material. Despite the high amount of La, no extra peak was found for the La-coated ceramic material, indicating the amorphous or poorly crystalline nature of the La coating.

### Example 6

### Arsenic Removal Performance of La-Coated Ceramic Material POU Filtration

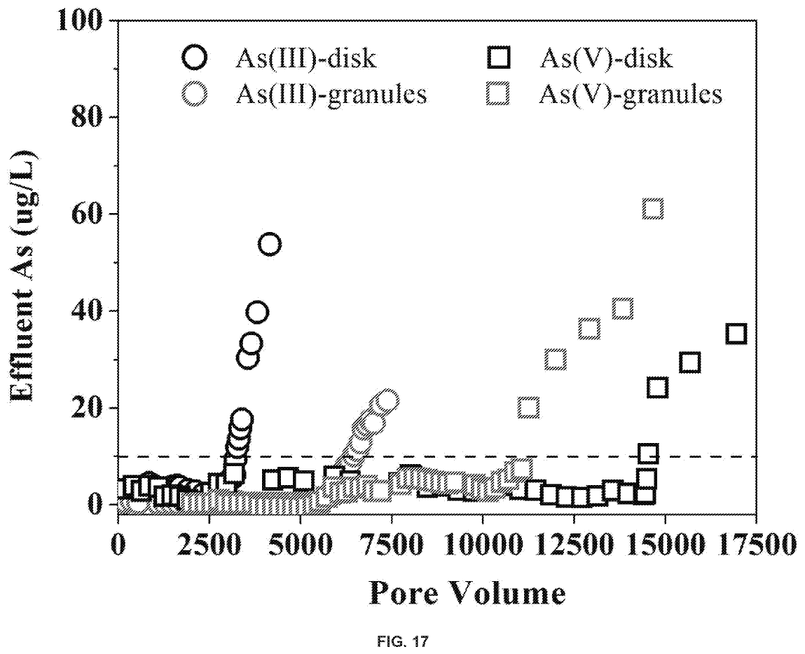

As(V) and As(III) removal was investigated using two types of representative prototype filters to simulate the performance of La-coated ceramic material in POU drinking water treatment applications. The La-coated ceramic disk filter was able to effectively treat ˜14,500 and ˜3,200 pore volumes (PVs) of water polluted by As(V) (0.120 mg/L) and As(III) (0.125 mg/L) below the MCL (0.010 mg/L), respectively (FIG. 17). Pore volume is the ratio between the volume of water that has passed the filter and the volume of the filter. In an earlier attempt, various iron-coated ceramic disk filters were developed and the best-performed filter could treat both As(V) and As(III) for ˜1200 PVs before breakthrough (Robbins, University of Kansas, Lawrence, K S, 2011). The La-coated ceramic disk filter developed in this study thus showed a significantly improved performance for both As(V) and As(III) removal, compared to the iron-coated material. Ceramic disk filters are frequently used in lab-scale investigations to simulate various full-scale POU devices such as ceramic pot, tubular and candle filters, which are commonly deployed in the field (Sobsey et al., Environ. Sci. Technol. 2008, 42, 4261-4267; Rayner et al., ACS Sustain. Chem. Eng. 2013, 1, 737-745; Perez-Vidal et al., Water Res. 2016, 98, 176-182; Wegmann et al., Water Res 2008, 42, 1726-1734). On a comparable basis, the equivalent treatment capacities were estimated to be 4.2×10⁴ and 9.3×10₃ L/m² (external surface) for As(V)- and As(III)-polluted water under similar conditions by the full-scale filters made from La-coated ceramic materials, respectively, which suggested that a full-size pot filter could potentially provide arsenic-safe drinking water to a family of four for about 2.6 and 0.6 years, respectively.

In addition, the bench-scale column filter packed with La-coated ceramic granules could treat ˜11,200 and 6,400 PVs of As(V)- and As(III)-contaminated water before the effluent arsenic concentration exceeding 0.010 mg/L (FIG. 17). Due to the easy-to-separate property, granular materials are widely used as filter media in various drinking water treatment applications, such as the development of POU filters. Our result suggested that 1 kg of La-coated ceramic granules could treat ˜5,000 L and ˜3,000 L of water with high levels of As(V) and As(III) contamination under similar conditions, respectively. Based on the low material cost to fabricate La-coated ceramic filters (less than $2/kg), the treatment cost of As(V)- and As(III)-contaminated water would be much less than $0.001/L for both disk and granule-packed column filters, which is considered high viable, cost-effective, and sustainable for POU household drinking water treatment, especially for developing countries (Sobsey et al., Environ. Sci. Technol. 2008, 42, 4261-4267). Notably, the La-coated ceramic filters also exhibited satisfactory stability that <0.02 wt % of the total La was leached from the La-coated ceramic filters into the solution throughout the filtration experiments under the experimental conditions.

### Example 7

### Arsenic Removal Performance of La-Coated Ceramic Material Batch Adsorption

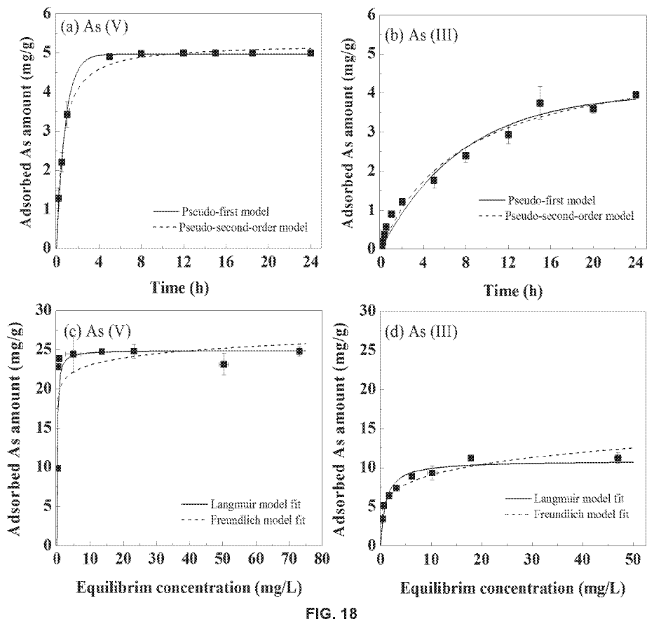

To fully investigate the sorption behavior of As(V) and As(III), batch adsorption experiments were performed to determine their adsorption kinetics and isotherms using La-coated ceramic granules. The kinetic curves of both As(V) and As(III) showed rapid initial uptakes, and the sorption reached equilibrium within 16 h (FIG. 18A-18B). The kinetics data were fitted with both pseudo-first order and pseudo-second order adsorption kinetics models, according to Equations (9) and (10) (Ho et al., Process Biochem. 1999, 34, 451-465), respectively. Where qₑ and q represent the quantities of adsorbed arsenic (mg/g) at equilibrium and at time t (h), respectively; and k₁ (h−1) and k₂ (g·mg⁻¹·h⁻¹) are the rate constants for pseudo-first order and pseudo-second order kinetic models, respectively. Both As(V) and As(III) adsorption kinetics were slightly better fitted by the pseudo-second order model than the pseudo-first order model (TABLE 5), suggesting that the chemisorption step was likely the rate-controlling step for arsenic sorption by La-coated ceramic materials (Ho et al., Process Biochem. 1999, 34, 451-465). Adsorption isotherms of As(V) and As(III) were obtained and fitted by both Langmuir (Equation 14) and Freundlich (Equation 15) models to determine their adsorption capacity (FIG. 18C-18D) (Langmuir, J. Am. Chem. Soc. 1918, 40, 1361-1403; Freundlich, Zeitschrift für Physikalische Chemie 1906, 57, 385-470).

$$\begin{matrix}{q}_{e} = \frac{{q}_{m} b {C}_{e}}{1 + b {C}_{e}} & ( 14 ) \\ {q}_{e} = {kC}_{e}^{1 / n} & ( 15 )\end{matrix}$$

Where Cₑ (mg/L) represents the equilibrium concentration of arsenic in solution, and qₑ (mg/g) stands for the amount of adsorbed arsenic per unit mass of adsorbent. In Equation (11), K represents the Langmuir equilibrium constant related to the energy of adsorption (Guo et al., Environ. Sci. Technol. 2005, 39, 6808-6818). For the Freundlich model, k represents the adsorption affinity, and n is an indicator associated with the adsorbent surface heterogeneity.

TABLE 5
Kinetics curve fitting parameters for adsorption of As(V)
and As(III) on La-coated ceramic material at pH 6.0.

| | pseudo-first order kinetic model | | | pseudo-second order kinetic model | | |
|---|---|---|---|---|---|---|
| | kₗ | qₑ^{b} | | k₂ | qₑ^{b} | |
| contaminant | (h⁻¹) | (mg · g⁻¹) | r² | (g · mg⁻¹ · h⁻¹) | (mg · g⁻¹) | r² |
| As (V) | 1.23 ± 0.07 | 4.97 ± 0.06 | 0.989 | 0.334 ± 0.031 | 5.24 ± 0.73 | 0.990 |
| As (III) | 0.130 ± 0.023 | 4.02 ± 0.26 | 0.970 | 0.0250 ± 0.007 | 5.14 ± 0.43 | 0.976 |

Both As(V) and As(III) adsorption isotherms could be better described with the Langmuir model (TABLE 6), and the Langmuir equilibrium constant for As(V) was >30 times higher than that for As(III), suggesting that the La-coated ceramic materials had a significantly higher adsorption affinity with As(V). Based on the Langmuir model, the estimated adsorption capacities for As(V) and As(III) were 24.8±0.1 and 10.9±0.5 mg/g under the experimental condition, respectively. The La-coated ceramic material exhibited superior performance for both As(V) and As(III) removal among the reported low-cost ceramic/clay-based sorbents suitable for POU applications (TABLE 7). Particularly, the La-coated ceramic material increased As(V) adsorption capacity by at least 3 folds in comparison to iron-modified ceramic tablets and porous sorbents (As(III) removal was not reported for these materials) (Chen et al., Desalination 2012, 286, 56-62; Chen et al., Colloids Surf. A 2012, 414, 393-399). Compared to surfactant-amended clay, the adsorption capacities of La-coated ceramic material for As(V) and As(III) increased by >30 folds (Li et al., Microporous Mesoporous Mater. 2007, 105, 291-297). While similar As(V) and As(III) adsorption capacities were reported for powdered activated alumina impregnated with La (Shi et al., ACS Appl. Mater. Interfaces 2015, 7, 26735-26741), the large size of La-coated ceramic granules prepared in the present work made it much easier to separate from the treated solution than powdered sorbents. Thus, the La-coated ceramic material developed in this study holds great promises as low-cost media for POU applications to remove both As(V) and As(III), especially for the developing countries with high levels of arsenic in drink water.

TABLE 6
Isotherm fitting parameters for adsorption of As(V)
and As(III) on coated ceramic material at pH 6.0.

| | Langmuir model | | | Freundlich model | | |
|---|---|---|---|---|---|---|
| contaminant | qₘ(mg · g⁻¹) | b(L · mg⁻¹) | r² | k | n | r² |
| As (V) | 24.8 ± 0.1 | 35.9 ± 2.8 | 0.973 | 23.7 ± 0.2 | 72.7 ± 17.9 | 0.761 |
| As (III) | 10.9 ± 0.5 | 1.01 ± 0.22 | 0.919 | 5.71 ± 0.41 | 5.00 ± 0.67 | 0.904 |

TABLE 7
Comparison of adsorption of arsenic on various adsorbents.

| | solu- | Max adsorption | | |
|---|---|---|---|---|
| | tion | capacity (mg/g) | | |
| Adsorbents | pH | As (V) | As (III) | reference |
| La-modified ceramics | 6.0 | 24.8 | 10.9 | this work |
| La-impregnated silica | 7.0 | 3.75 | —^{a} | Wasay et al., 1996 |
| gel | | | | |
| iron-impregnated | 6.9 | 8.49 | — | Chen, Zhang et al., |
| table ceramic | | | | 2012 |
| iron-impregnated | 6.9 | 7.12 | — | Chen, Lei et al., 2012 |
| porous ceramic | | | | |
| surfactant-modified | 7 | 0.674 | 0.322 | Li et al., 2007 |
| kaolinite | | | | |
| magnetic wheat straw | — | 8.062 | 3.898 | Tian et al., 2011 |
| La-impregnated | 7.0 | 26.3 | 9.23 | Shi et al., 2015 |
| activated alumina | | | | |

^{a}Not mentioned

### Example 8

### Effects of Solution pH and Coexisting Ions on Arsenic Sorption

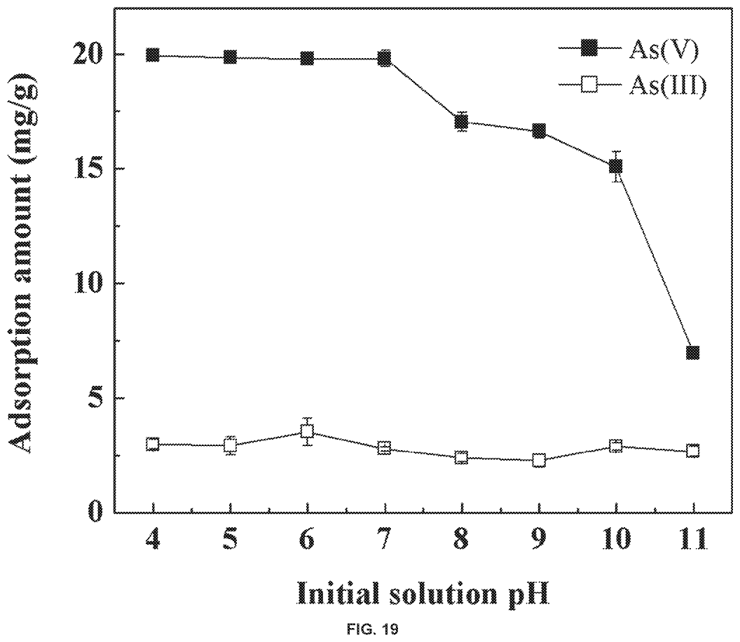

Solution pH had different impacts on the sorption of As(V) and As(III) (FIG. 19). A clear pH dependency was observed for As(V) sorption on La-coated ceramic materials. The materials exhibited superior performance under acidic and neutral conditions (pH 4-7) with a removal efficiency ≥99/for As(V). Increasing the pH gradually decreased the material performance under alkaline conditions with the sorption amount dropping to ˜7 mg/g at pH 11, which is about one third of that at pH 4. Similar trends were observed for As(V) adsorption on various metal (hydr)oxide sorbents (e.g., Cu/Mg/Fe/La Layered double hydroxide, Fe—La (hydr)oxides), which may be due to the change of the sorbents' surface sites that impacted their interaction with anionic contaminants (Guo et al., J. Hazard. Mater. 2012, 239, 279-288; Zhang et al., Chem. Eng. J. 2014, 251, 69-79). Notably, although As(V) sorption decreased with increasing pH, the La-modified ceramic materials still showed high As(V) sorption amount (>17 mg/g) under circumneutral conditions that are most relevant to natural aquatic systems (pH 5-8). Compared to As(V), As(III) sorption was much less sensitive to solution pH that similar removal efficiency was observed from 4 to 11. Zhang et al. reported a similar trend for As(III) adsorption on bimetal oxide materials and they attributed that to the uncharged nature of H₃AsO₃ under various pH conditions (Zhang et al., Chem. Eng. J. 2010, 158, 599-607).

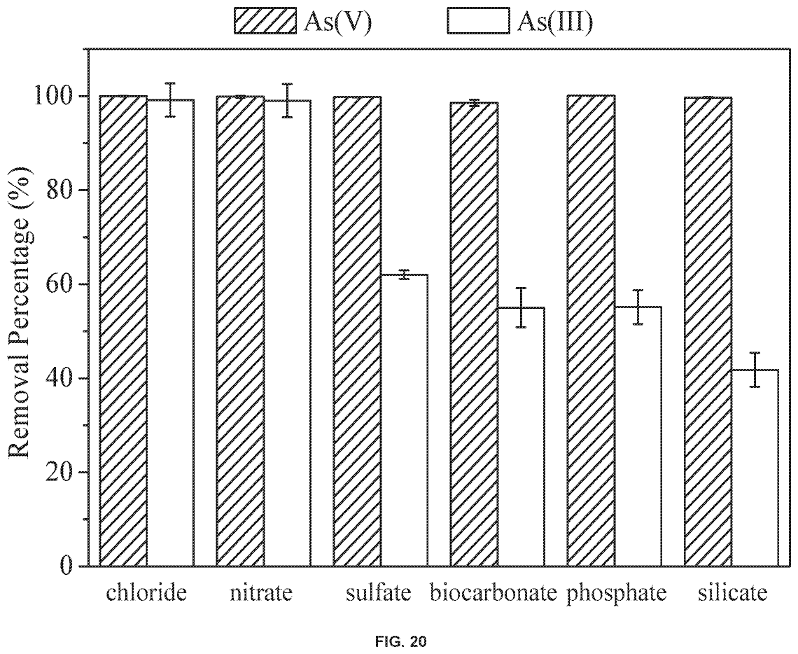

The sorption of As(V) was less influenced by coexisting anions than that of As(III). As shown in TABLE 8, the presence of coexisting anions generally had minimal effects on the removal of As(V) under the experimental conditions, except for bicarbonate where a more noticeable inhibition was observed with increasing concentrations. Compared to the bicarbonate-free system, As(V) sorption decreased by 25.8% and 47.3% when bicarbonate concentrations increased to 30.5 (0.5 mM) and 305 mg/L (5 mM), respectively, probably due to the competition between carbonate and As(V) for the surface sites of La coating (Anawar et al., Chemosphere 2004, 54, 753-762). Similar inhibitory effects were reported on various metal oxide-based sorbents (e.g., Fe—Mn binary oxide-impregnated chitosan granular bead (Qi et al., Bioresour. Technol. 2015, 193, 243-249) and Ce—Ti oxide adsorbent (Deng et al., J. Hazard. Mater. 2010, 179, 1014-1021)) in the presence of bicarbonate (>61.0 mg/L). For As(11) sorption, while chloride and nitrate displayed negligible effects under the experimental concentrations, more significant inhibitory effects were observed in the presence of sulfate, bicarbonate, silicate and phosphate, and the adsorbed amount of As (III) decreased with increasing concentrations of the coexisting anions (TABLE 8). Similar trend was observed with the use of a low arsenic concentration (0.120 mg/L, FIG. 20). Results suggested that coexisting anions generally had a stronger inhibition on As(III) than As(V) removal, indicating that these two processes were likely governed by different mechanisms.

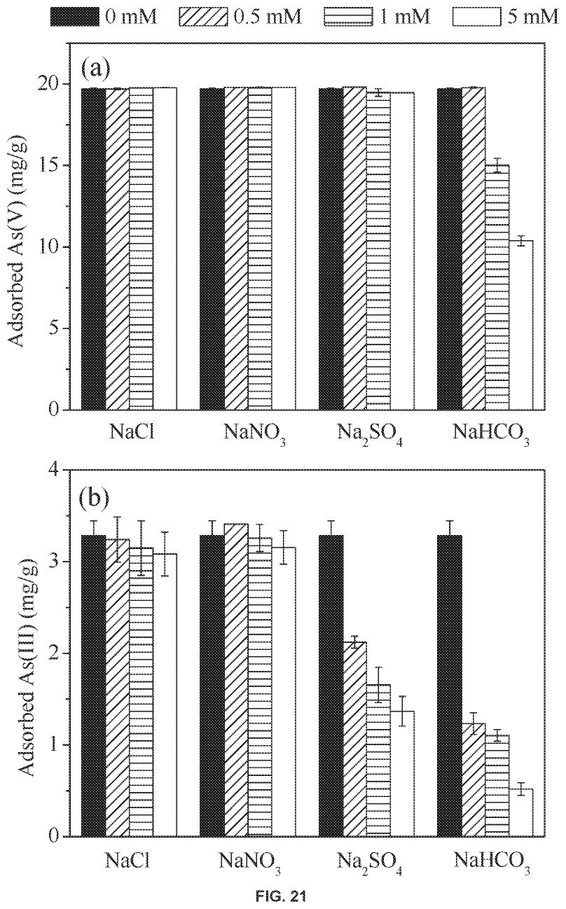

The influence of coexisting anions (Cl⁻, NO₃⁻, SO₄²⁻ and HCO₃⁻) on both As(V) and As(III) adsorption by La-coated ceramic granules were investigated at the typical concentration range of natural water (0-5 mM). The corresponding results were shown in FIG. 21. The presence of Cl⁻, NO₃⁻, and SO₄²⁻ had little effects on the removal of As(V), which were observed at all examined concentrations. At the same time, HCO₃⁻ also did not affect As(V) removal with 0.5 mM concentration. When HCO₃⁻ concentration increased to 1 and 5 mM, As(V) adsorption decreased to 76.2% and 52.7% as the adsorption amount in HCO₃⁻-free condition. HCOs had an inhibitory effect on As(V) adsorption and this inhibition effect was more significant at higher concentration. Reduction of As(V) removal have also been reported on Fe—Mn binary oxide impregnated chitosan granular bead (Qi et al., Bioresource Technology 2015, 193, 243-249) and Ce—Ti oxide adsorbent (Deng et al., Journal of Hazardous Materials 2010, 179, 1014-1021) in the presence of HCO₃⁻ with >1 mM concentration. For As(III) adsorption, Cl⁻ and NO₃⁻ also posed little effects in the examined concentration range. Meanwhile, interfering effects of SO₄⁻ and HCO₃⁻ on As(III) adsorption were significant and the adsorbed amount of As (III) were decreased with higher SO₄²⁻/HCO₃⁻ concentrations. Bimetal oxide magnetic nanomaterials MnF₂O₄ and CoFe₂O₄ were also reported to have a worse As(III) removal permeance with HCO₃⁻ in the range of 0.1-10 mM concentrations in neutral solutions (Zhang et al., Chemical Engineering Journal 2010, 158, 599-607). Above results on the arsenic removal in the presence of coexisting ions suggested that side effects of coexisting anions on As(TH) adsorption were much more significant than that on As(V) adsorption.

TABLE 8
Effects of coexisting anions on As(V) and As(III) sorption
by La-coated ceramic materials at pH 6. The As(V) and
As(III) concentrations are 20 mg/L and 5 mg/L, respectively,
and the sorbent dosage is 1.0 g/L.

| | | | Adsorbed arsenic amount (mg/g) | |
|---|---|---|---|---|
| coexisting anions | | | As(V) | As(III) |
| Chloride | 0 | mg/L | 19.9 ± 0.1 | 3.28 ± 0.16 |
| | 17.7 | mg/L (0.5 mM) | 19.8 ± 0.1 | 3.24 ± 0.25 |
| | 35.5 | mg/L (1 mM) | 19.9 ± 0.1 | 3.15 ± 0.30 |
| | 177 | mg/L (5 mM) | 19.9 ± 0.1 | 3.52 ± 0.58 |
| Nitrate | 31.0 | mg/L (0.5 mM) | 20.0 ± 0.1 | 3.41 ± 0.01 |
| | 62.0 | mg/L (1 mM) | 19.9 ± 0.1 | 3.26 ± 0.15 |
| | 310 | mg/L (5 mM) | 20.0 ± 0.1 | 3.15 ± 0.18 |
| Sulfate | 48.0 | mg/L (0.5 mM) | 19.9 ± 0.1 | 2.12 ± 0.06 |
| | 96.1 | mg/L (1 mM) | 19.8 ± 0.1 | 1.65 ± 0.19 |
| | 480 | mg/L (5 mM) | 19.8 ± 0.1 | 1.37 ± 0.16 |
| Bicarbonate | 30.5 | mg/L (0.5 mM) | 19.9 ± 0.1 | 1.23 ± 0.12 |
| | 61.0 | mg/L (1 mM) | 15.0 ± 0.4 | 1.10 ± 0.06 |
| | 305 | mg/L (5 mM) | 10.4 ± 0.3 | 0.51 ± 0.07 |
| Silicate | 7.6 | mg/L (0.1 mM) | 20.0 ± 0.1 | 2.21 ± 0.21 |
| | 38.0 | mg/L (0.5 mM) | 20.0 ± 0.1 | 1.19 ± 0.06 |
| | 76.1 | mg/L (1 mM) | 19.9 ± 0.1 | 1.22 ± 0.06 |
| Phosphate | 0.960 | mg/L (0.01 mM) | 20.0 ± 0.1 | 2.79 ± 0.07 |
| | 4.80 | mg/L (0.05 mM) | 19.9 ± 0.1 | 2.74 ± 0.15 |
| | 9.60 | mg/L (0.1 mM) | 19.9 ± 0.1 | 2.32 ± 0.10 |

### Example 9

### Possible Sorption Mechanisms

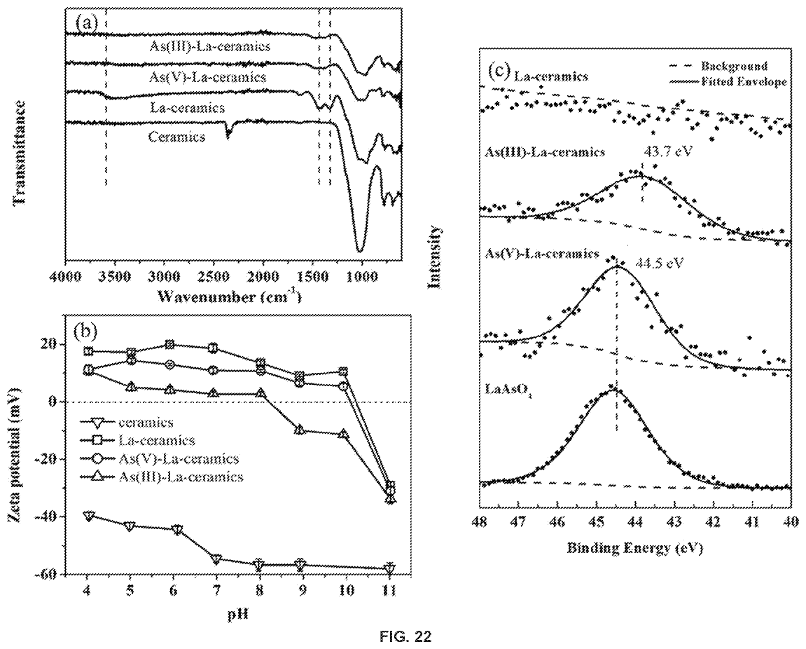

To investigate the removal mechanisms of As(V) and As(III), FTIR spectra of La-coated ceramic material before and after arsenic sorption were obtained to determine the La surface functional group(s) that might be involved in As(V) and As(III) sorption. Compared to the raw ceramic material without coating, the new peaks at 3554, 1450 and 1300 cm⁻¹ for La-coated ceramic material corresponded to the O—H stretching group of La (hydr)oxide, stretching vibration of H—O—H, and vibration mode of NO₃⁻, respectively (FIG. 22A) (Jais et al., Sep. Purif. Technol. 2016, 169, 93-102; Zhang et al., Chem. Eng. J. 2012, 185, 160-167). Additionally, the broader and less intense peak at ˜1000 cm⁻¹ could be attributed to the combination bands of La-0 fundamental vibrational modes and Si—O—Si stretch (Jang et al., Microporous Mesoporous Mater. 2004, 75, 159-168). Results indicated that LaONO₃/LaOOH might be the predominant La phases on the ceramic surface when coated at 385° C. (Gobichon et al., Solid State Tonics 1996, 93, 51-64). After As(V) and As(II) sorption, the peaks at 3554, 1450 and 1300 cm¹ were significantly reduced or even disappeared, suggesting that these functional groups might play a role in the sorption of As(V) and As(III). Previous studies also reported the important role of hydroxyl group induced by La modification in anion adsorption by La-coated red mud and alumina (Cui et al., PLOS ONE 2016, 11, e0161780; Shi et al., J. Mater. Chem. A 2013, 1, 12797-12803). Meanwhile, the combination bands of La-0 fundamental vibrational modes and Si—O—Si stretch (˜1000 cm⁻¹) became broader and less intense after As(V) and As(III) sorption, which was probably affected by the broad overlapping peaks of As—O band in this region (Li et al., Chem. Eng. J. 2010, 161, 106-113). Results suggested that sorption of both As(V) and As(UI) could be related to the functional groups of LaOOH/LaONO₃ that existed on the ceramic material surface. Control experiments found that the uncoated ceramic material had sorption capacities of 1.13±0.27 mg/g and 0.096±0.022 mg/g for As(V) and As(III), respectively. Compared to the uncoated ceramic material, the La-coated ceramic materials increased the As(V) and As(III) sorption capacities by >20 folds and >100 folds, respectively (FIG. 18C-18D). Results further suggested the crucial role of La coating in arsenic removal.

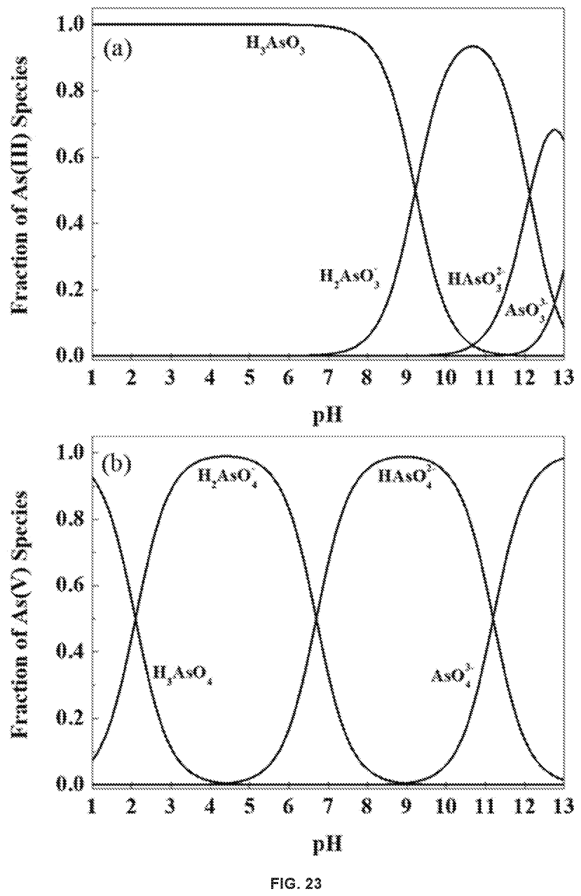

Zeta potential measurements were performed to determine the surface charge of the La-coated ceramic material prior to and after arsenic sorption. The La-coated ceramic material had a high point of zero charge pH (pHPZC) of ˜10.2, suggesting that the material surface was positively charged under a range of pH conditions (FIG. 22B). The predominant species of As(V) were negatively charged (i.e., H₂AsO₄⁻ and HAsO₄²⁻) under the experimental pH conditions (FIG. 23), and thus the positively charged sorbent surface would favor the removal of As(V) anions through electrostatic attraction. For As(III), the uncharged H₃AsO₃ was predominant at pH<9.2 (Raven et al., Environ. Sci. Technol. 1998, 32, 344-349). Therefore, electrostatic interaction would have a minimal impact on As(III) sorption by the La-coated ceramic material, which is consistent with the observation that As(III) sorption was insensitive to the solution pH that affected the surface charge of the sorbent (FIG. 19). After sorption, the pHPZC of the As(III)-loaded La-coated ceramic material was shifted to more acidic values (i.e., pH ˜8.2) (FIG. 22B), suggesting the formation of inner-sphere surface complexes between As(III) and the sorbent surface (Pena et al., Environ. Sci. Technol. 2006, 40, 1257-1262). In contrast, while sorption of As(V) slightly reduced the surface charge of the La-coated ceramic material, the pHPZC of the sorbent practically remained the same after sorption, indicating that formation of inner-sphere surface complexes might not be the primary mechanism for As(V) removal. It should be noted that based on the adsorption isotherm study (FIG. 18C-18D), the La-coated ceramic material appeared to exhibit a much higher affinity with As(V) than As(III), but electrostatic attraction would primarily form loosely bonded outer-sphere surface complexes (Catalano et al., Geochim Cosmochim Ac. 2008, 72, 1986-2004). Our results thus suggested that mechanisms other than electrostatic interaction would also contribute significantly to the sorption of As(V).

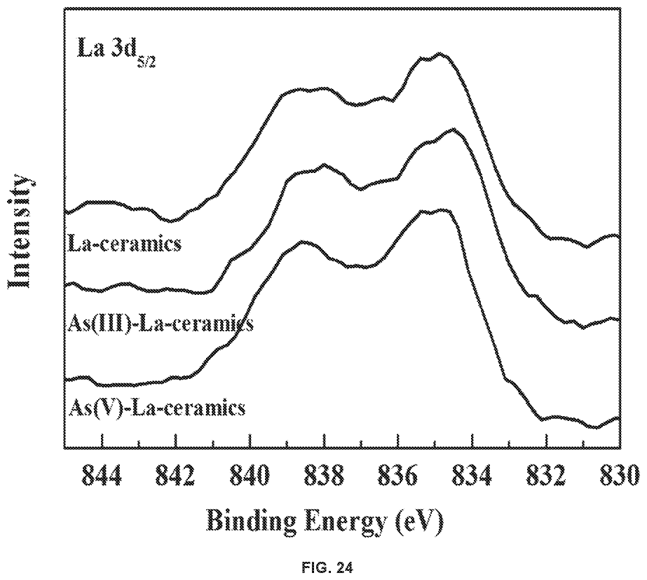

To further understand the interaction between arsenic and the La-coated ceramic material, XPS spectra of the sorbents before and after As(V) and As(III) sorption were collected and analyzed. Compared to the raw La-coated ceramic material, new and strong peaks appeared in the As 3d spectra after the sorption of As(V) and As(III), clearly confirming the presence of arsenic and its successful binding to the sorbent surface (FIG. 22C). Notably, only one peak could be assigned to each spectrum after As(V) or As(III) sorption. Suggesting that their oxidation states did not change during the sorption process. Although there was no report in literature on the binding energy of arsenic adsorbed onto La-based materials, the binding energy of As(V) is usually 0.7-1.3 eV higher than that of As(III) (Ding et al., Chemosphere 2017, 169, 297-307; Ouvrard et al., Geochim Cosmochim Ac. 2005, 69, 2715-2724; Zhang et al., Environ. Sci. Technol. 2017, 51, 6326-6334), and thus the binding energies at 44.4 eV and 43.7 eV could be assigned to As(V) and As(III), respectively. As a result, it suggested oxidation states of As(V) and As(III) on the ceramic surface did not change during the sorption process. The observed binding energies for both As(V) and As(III) were lower than those adsorbed onto other metal oxides (FIG. 24) (Ding et al., Chemosphere 2017, 169, 297-307; Cheng et al., Water Res. 2016, 96, 22-31), indicating the strong interaction between arsenic and the La surface that might alter the coordination environments of As(V) and As(III). Additionally, the As 3d spectrum of pure LaAsO₄ was also obtained as a reference, and the As(V) binding energy was in close agreement to the As(V)-loaded La-coated ceramic material (FIG. 22C). Because of the similar As(V) coordination environments, the results indicated that LaAsO₄ surface precipitates might form on the La-coated ceramic material after As(V) sorption. Compared to inner-sphere surface complexes, formation of surface precipitates has been reported to exhibit a much less pronounced effect on the sorbent surface charge (Li et al., J. Colloid Interf. Sci. 2000, 230, 12-21), which is consistent with the similar zeta potentials observed for La-coated ceramic materials before and after As(V) sorption in the present work (FIG. 22B).

Based on the characterization of La-coated ceramic materials before and after As(V) and As(III) sorption, as well as the sorption behavior of As(III) and As(V) under different water chemistry parameters, we proposed that As(V) removal might be primarily attributed to the formation of insoluble LaAsO₄ surface precipitates, and electrostatic interaction between As(V) and La-coated ceramic surface might also play a minor role. In contrast, As(III) was predominantly removed by the formation of inner-sphere complexes with La component on the ceramic surface. The exact binding nature and coordination environments between As(V)/As(III) and La-coated ceramic material worth further investigation.

### Example 10

### Characterization of Ceramic Filters Made of Different Combustible Materials

Ceramic filters made of different combustible materials exhibited different pore properties and morphologies (FIG. 25 and TABLE). In general, when the same ratio of combustible material was used, the porosity of the ceramic filters followed the order of starch >rice husk >cellulose fiber, and the order of average pore size was cellulose fiber >rice husk≈starch. For each type of combustible material, both porosity and average pore diameter of the ceramic filters increased with a higher combustible material mixing ratio, due to the increased amount of the combustible materials burnt out during the firing process. For instance, the porosity increased from 15.0% to 39.1% when the mixing ratio of cellulose fiber increased from 10% to 30%.

TABLE 9
Properties of different ceramic filters.

| Filter types | | | | Total pore | Average |
|---|---|---|---|---|---|
| Combustible | | Density | Porosity | area | pore size^{a} |
| material | percentage | (g/mL) | (%) | (m²/g) | (μm) |
| cellulose | 10% | 1.07 | 15.0 | 0.964 | 0.926 |
| fiber | 15% | 0.917 | 21.7 | 1.09 | 1.22 |
| | 20% | 0.736 | 23.7 | 1.58 | 1.24 |
| | 30% | 0.528 | 39.1 | 1.29 | 2.24 |
| rice husk | 10% | 1.07 | 22.2 | 1.39 | 0.602 |
| | 15% | 0.913 | 33.2 | 2.08 | 0.701 |
| | 20% | 0.760 | 31.0 | 2.44 | 0.965 |
| starch | 10% | 1.13 | 26.3 | 1.84 | 0.595 |
| | 15% | 0.900 | 28.0 | 1.54 | 0.810 |
| | 20% | 0.902 | 34.3 | 1.43 | 1.07 |
| | 30% | 0.688 | 35.2 | 1.23 | 1.26 |

^{a}The average pore diameter was obtained by 4 V/S, where V is the total pore volume and S the surface area by MIP.

The selection of combustible materials led to significant differences in the pore size distribution of the ceramic filters (FIG. 25D-25F). A single peak was observed in the pore size distribution of ceramic filters fabricated using starch (starch filter for short); in contrast, filters made of rice husk (rice-husk filter for short) showed a bimodal pore size distribution pattern containing two separate peaks at pore sizes around 0.41-0.68 μm and 19.8-24.1 μm, respectively. Meanwhile, filters made of cellulose fiber (cellulose fiber filter for short) displayed a different pore size distribution pattern from starch or rice-husk filters, showing a bimodal pore size distribution pattern without significant separated peaks (FIG. 25D-25F). When increasing the combustible material mixing ratio, the relative abundance of large pores was increased. For example, when the starch mixing ratio increased from 10% to 30%, the filter peak pore size shifted from 1.0 μm to 3.7 μm, indicating an increase of large pores; at the same time, a uniform single-peak pore size distribution was maintained. Kallman and Smith investigated the pore size distribution of ceramic filters using sawdust as combustible material and observed a similar phenomenon that the percentage of small pores decreased with an increasing sawdust mixing ratio (Kallman et al., J. Environ. Eng.-ASCE 2011, 137, 407-415). It was noteworthy that the mixing ratio of combustible material did not alter the pore size distribution patterns of ceramic filters made of each combustible material. The different pore size distribution patterns may probably be related to the distinct morphological properties of the combustible materials (FIG. 26).

XRF was applied to determine the chemical composition of the ceramic filters. SiO₂, Al₂O₃, and Fe₂O₃ were identified as the main composition of the ceramic filters (TABLE 10). Small amounts (<5%) of K₂O, MgO, TiO₂ and CaO were also observed in the ceramic filters. It was worth noting that despite the type and ratio of the combustible material, the elemental contents of all filters were quite similar. Results clearly suggested that the combustible material had negligible impact on the chemical composition of the ceramic filters.

TABLE 10
Major elements of ceramic filters measured by XRF.

| | Cellulose fiber | | | | Rice husk | | | Starch | | | |
|---|---|---|---|---|---|---|---|---|---|---|---|
| | 10% | 15% | 20% | 30% | 10% | 15% | 20% | 10% | 15% | 20% | 30% |
| SiO₂ | 64.45 | 63.90 | 63.58 | 63.01 | 65.12 | 64.70 | 64.73 | 64.73 | 64.73 | 64.73 | 64.32 |
| Al₂O₃ | 17.86 | 17.76 | 17.95 | 17.99 | 17.42 | 17.45 | 17.40 | 17.40 | 17.40 | 17.40 | 17.53 |
| Fe₂O₃ | 7.59 | 7.58 | 7.51 | 7.37 | 7.55 | 7.56 | 7.55 | 7.55 | 7.55 | 7.55 | 7.68 |
| K₂O | 4.13 | 4.10 | 4.10 | 3.97 | 4.13 | 4.12 | 4.10 | 4.10 | 4.10 | 4.10 | 4.18 |
| MgO | 1.51 | 1.50 | 1.51 | 1.47 | 1.54 | 1.56 | 1.55 | 1.55 | 1.55 | 1.55 | 1.55 |
| TiO₂ | 1.12 | 1.10 | 1.13 | 1.13 | 1.09 | 1.08 | 1.08 | 1.08 | 1.08 | 1.08 | 1.09 |
| CaO | 0.42 | 0.52 | 0.65 | 0.93 | 0.32 | 0.43 | 0.42 | 0.42 | 0.42 | 0.42 | 0.36 |
| P₂O₅ | 0.14 | 0.20 | 0.12 | 0.15 | 0.13 | 0.13 | 0.14 | 0.14 | 0.14 | 0.14 | 0.14 |
| Na₂O | 0.08 | 0.08 | 0.08 | 0.08 | 0.09 | 0.09 | 0.09 | 0.09 | 0.09 | 0.09 | 0.14 |
| LOI | 0.84 | 0.86 | 0.87 | 1.26 | 0.49 | 0.76 | 0.93 | 0.93 | 0.93 | 0.93 | 0.87 |

All results reported as wt. %

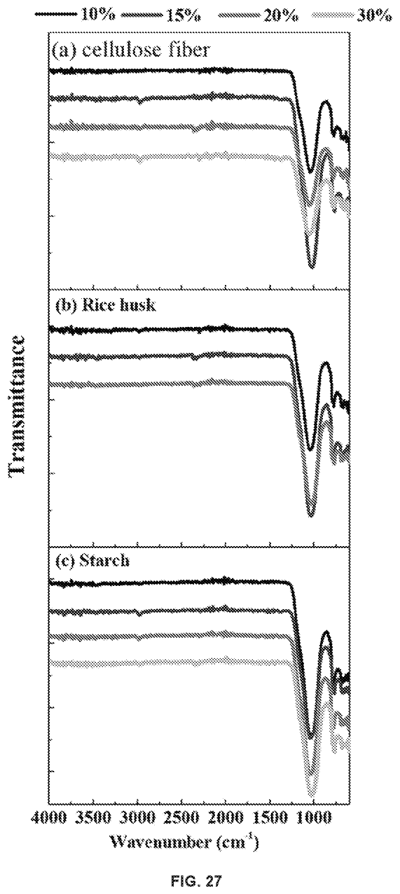

FTIR spectra were obtained to further determine the major functional groups of the ceramic filters. Peaks at 1030 cm⁻¹, 780 cm⁻¹ and 667 cm⁻¹ were observed for all ceramic filters (FIG. 27), corresponding to the concerted Si—O—Si, Si—O—Al and Fe—O stretches, respectively (Nayak et al., Bull. Mater. Sci. 2007, 30, 235-238; Jang et al., Microporous Mesoporous Mater 2004, 75, 159-168). Results were consistent with the XRF analysis showing the predominance of SiO₂, Al₂O₃, and Fe₂O₃ in the ceramic filters. The lack of carbon associated bands suggested that all combustible materials were completely burnt out during the firing process. Additionally, similar spectra were observed for all ceramic filters regardless of the type and ratio of the combustible material, indicating the presence of similar functional groups for all filters. Combined, the characterization results suggested that the combustible material had a strong influence on the porosity and pore size distribution of the ceramic filters, while they had a minimal impact on the ceramic filter composition and surface functionality.

### Example 11

### Filtration Performance

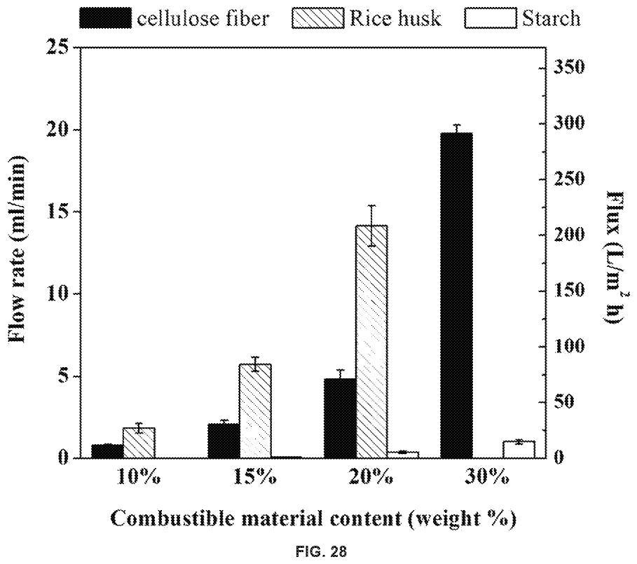

FIG. 28 showed the flow rate of clean ceramic disk filters. The flow rates were strongly influenced by both the type of combustible materials and their mixing ratio. For the same type of combustible material, the flow rate increased significantly with a higher mixing ratio. Specifically, the average flow rate of the filters made of the highest ratios of cellulose fiber, rice husk and starch increased 25, 7.7 and 13 times than those made of the lowest ratio of the corresponding combustible material, respectively. The higher flow rates may be ascribed to the large pore abundance of the filters resulting from the use of more combustible materials. Similar trends were observed for ceramic pot filters made of different amounts of rice husk by RDIC factory in Cambodia, showing that an increase of the rick husk amount by 45% resulted in up to 5.3 times increase of the flow rate of the pot filters (Van der Laan et al., Water Res. 2014, 51, 47-54; Van Halem et al., Water Res. 2017, 124, 398-406). With the same mixing ratio, the flow rate of the ceramic filters followed the order of rice husk >cellulose fiber >starch (FIG. 28). The difference of the flow rate may be due to the distinct pore size distribution of the filters prepared from different combustible materials. While the porosity generally decreased in the order of starch >rice husk >cellulose fiber, the use of rice husk produced a significant portion of large pores (i.e., peaks at 19.8-24.1 μm), resulting in a higher flow rate. In contrast, starch filters showed a uniform pore size distribution with a relatively small pore size (e.g., sharp peak at 2.0 μm for 20%-starch filter) (FIG. 25E-25F), which may cause a significantly slower flow rate. In a previous study, Oyanedel-Craver and Smith found that ceramic disk filters prepared from different soils exhibited similar porosities but significantly different flow rates, which were also attributed to the different pore size distribution of the filters (Oyanedel-Craver et al., Environ. Sci. Technol. 2008, 42, 927-933).

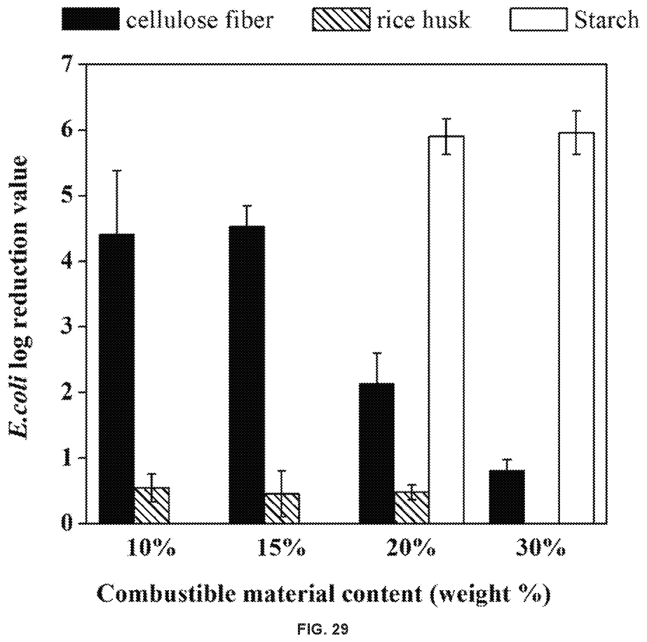

The bacterial removal efficiencies of the clean disk filters were shown in FIG. 29. Similarly, the type of combustible material significantly impacted the bacterial removal efficiency. When starch was used as the combustible material, bacterial LRVs were as high as 5.90 and 5.96 for ceramic filters prepared with 20% and 30% of starch, respectively. Since the flow rates of 10%- and 15%-starch filters were extremely slow (<0.001 and 0.078 mL/min, respectively), their bacterial removal performances were not tested. Meanwhile, satisfactory bacterial LRVs (i.e., 2.1-4.5) were also observed for filters made of cellulose fiber with a mixing ratio of 10%-20%. Increasing the cellulose fiber ratio to 30% significantly decreased the bacterial LRV, which might be attributed to the sharp increase of the large pores (i.e., >20 μm) in the resulting filters (FIG. 25D). With the use of sawdust as the combustible material, Kallman et al. reported a similar trend that the bacterial LRV dropped by 2 with an increased sawdust ratio from 4% to 17%.6 Compared to starch and cellulose fiber, low LRVs (<1.0) were observed for all rice-husk filters (FIG. 29) from rice husk ration from 10% to 20%.

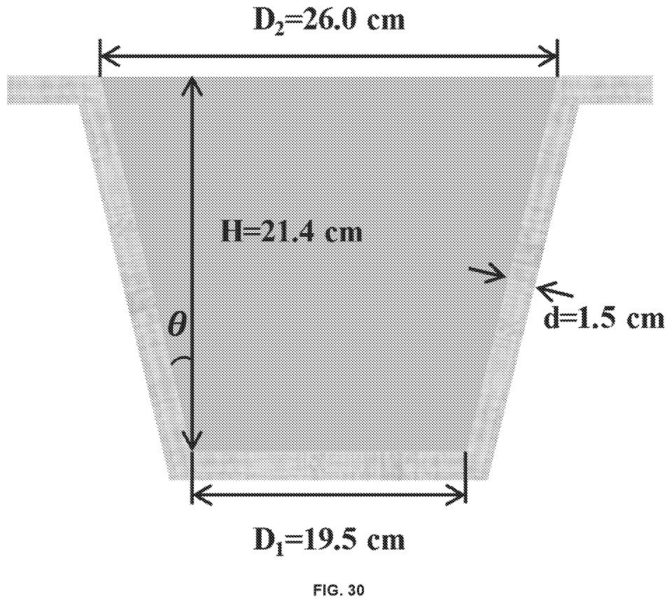

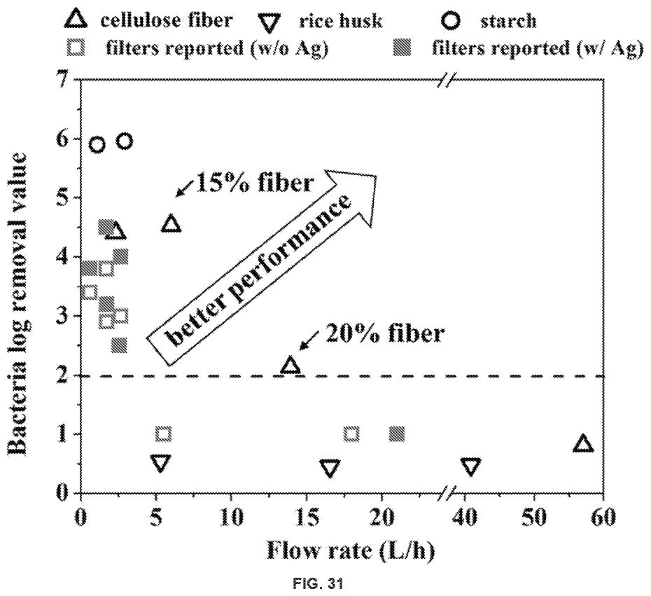

The performance of ceramic filters is primarily determined by two competing factors: flow rate and microbial removal efficiency, and filters that can achieve higher flow rate while maintaining effective microbial removal efficiency would be more desirable. To compare the filtration performance of disk filters to existing filters with different forms reported in previous research (e.g., pot filter), all flow rates were adjusted to the “equivalent” flow rate of the full-size ceramic pot filter with a frustum shape (FIG. 30) based on established methods (Schweitzer et al., Environ. Sci. Technol. 2012, 47, 429-435). FIG. 31 showed the equivalent flow rate and microbial removal efficiency of reported ceramic filters (with and without silver coating) (Van der Laan et al., Water Res. 2014, 51, 47-54; Oyanedel-Craver et al., Environ. Sci. Technol. 2008, 42, 927-933; Van Halem et al., Water Res. 2017, 124, 398-406; Bielefeldt et al., Water Res. 2009, 43, 3559-3565) as well as filters prepared in the present work. According to WHO, a microbial removal efficiency of 2 LRV or above is considered reaching the “protective” level of bacteria performance criteria (World Health Organization 2011). Within this constraint, the flow rates of existing ceramic pot filters were usually ≤3 L/h (Rayner et al., J Water Sanit. Hyg. De. 2013, 3, 252-261). In this research, the rice husk ceramic filters exhibited relatively high flow rates, but their bacterial removal efficiencies were low (i.e., LRV <1) and thus practically ineffective. The ceramic filters made of starch as the combustible material had high bacterial removal efficiency (i.e., LRV >5), but their flow rates were relatively low (i.e., ≤3 L/h).

Further, flow rate and microbial removal efficiency of ceramic filters before and after La modification were measured (TABLE 11-12). Filters tested contained 20% cellulose fiber (TABLE 11) or 30% cellulose fiber (TABLE 12) or 20% rice husk. The results showed that La-modification can increase bacterial removal efficiency but did not decrease flow rate significantly.

TABLE 11
Results of bacteria removal in La-modified ceramic
filter contained 20% cellulose fiber.

| | flow rate (mL/min) | | LRV | | |
|---|---|---|---|---|---|
| La | before | after | before | after | La component |
| modifi- | modifi- | modifi- | modifi- | modifi- | percentage in |
| cation | cation | cation | cation | cation | the disc (%) |
| 28 ml 1.2M | 5.46 | 3.51 | 1.74 | 5.82 | 11.4 |
| La(NO₃)₃ | | | | | |
| 28 ml 0.12M | 4.45 | 4.45 | 2.64 | 5.23 | 0.80 |
| La(NO₃)₃ | | | | | |

TABLE 12
Results of bacteria removal in La-modified ceramic
filter contained 30% cellulose fiber.

| | flow rate (mL/min) | | LRV | | La |
|---|---|---|---|---|---|
| La | before | after | before | after | component |
| modifi- | modifi- | modifi- | modifi- | modifi- | percentage in |
| cation | cation | cation | cation | cation | the disc (%) |
| 28 ml 1.2M | 34.0 | 25.0 | 0.31 | 3.21 | 8.99 |
| La(NO₃)₃ | | | | | |
| 28 ml 0.12M | 27.7 | 24.7 | 0.97 | 2.30 | 0.81 |
| La(NO₃)₃ | | | | | |

The performance of the ceramic filters made of cellulose fiber, especially when the mixing ratio was ≤20%, was significantly improved in terms of both flow rate and bacterial removal efficiency in comparison to existing ceramic filters (Van der Laan et al., Water Res. 2014, 51, 47-54; Oyanedel-Craver et al., Environ. Sci. Technol. 2008, 42, 927-933; Van Halem et al., Water Res. 2017, 124, 398-406; Bielefeldt et al., Water Res. 2009, 43, 3559-3565). Specifically, 15%-cellulose fiber filters could achieve >4 LRV bacteria removal (>99.99% removal), which was significantly higher than those of reported silver-free ceramic pot filters; the high microbial removal efficiency was even comparable to those using filters after impregnated with silver (Oyanedel-Craver et al., Environ. Sci. Technol. 2008, 42, 927-933; Bielefeldt et al., Water Res. 2009, 43, 3559-3565). Meanwhile, a high equivalent flow rate of 5.9 L/h was maintained. For 20%-cellulose fiber ceramic filters, the bacterial removal efficiency was higher than 2 LRV and the equivalent flow rate was 13.9 L/h which represented >3-fold increase compared to existing ceramic filters. Our results suggested that the use of cellulose fiber as the combustible material in ceramic filter manufacture has a great potential to significantly increase the flow rate of POU ceramic filter with effective bacterial removal.

Since all filters fabricated in the present work had similar composition and surface functional groups, the significantly different filtration performance among filters made of cellulose fiber, starch, and rice husk may primarily be attributed to their distinct pore size distribution patterns.

### Example 12

### Semi-Quantitative Modeling Results

The removal of microbial cells by the ceramic disk filters is primarily caused by physical straining, a process that occurs when the cells enter pores that are too small to allow their passage (Xu et al., Environ. Sci. Technol. 2008, 42, 771-778; Xu et al., Water Resour. Res. 2006, 42; Xu et al., Water Resour. Res. 2009, 45). Our control experiment found that the bacterial removal was <0.2 LRV by ceramic granules where the physical straining capture was limited, confirming that attachment capture was negligible for bacterial removal. In this research, we related the flow distribution within the pores of various sizes to the possibility of bacterial capture and presented a simple mathematical framework that could allow for the semi-quantitative analysis of microbial removal efficiency of ceramic disk filters based on the measured pore size distribution (Equations 3-8).

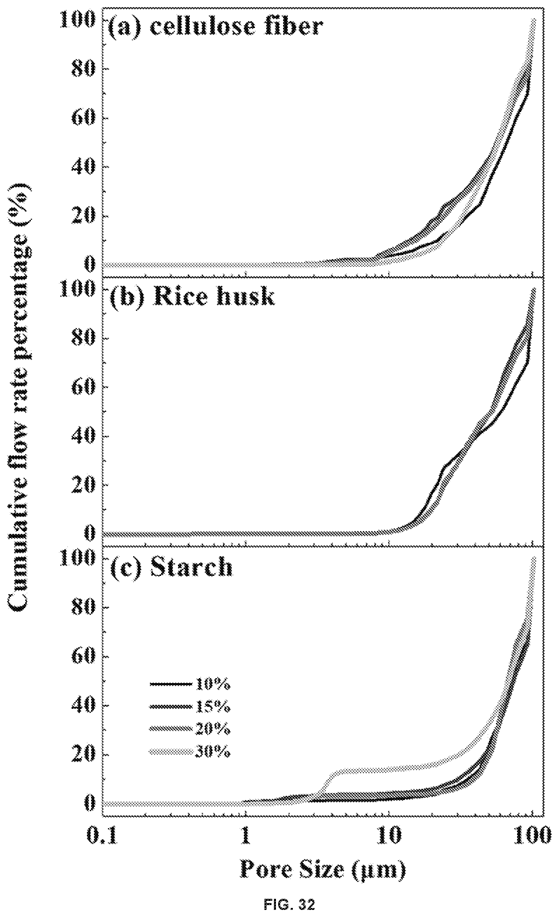

Based on the measured pore size distributions of the various ceramic disk filters, the corresponding flow rate distribution curves were calculated (FIG. 32), which, in essence, showed that as water preferentially flowed through the relatively large pores, only a small fraction of water flowed through pores that are smaller than the dimensions of the bacterial cells (TABLE 13), where the cells would be captured due to straining. The probability that water would flow through small pores (pores with sizes smaller than E. coli cells' size 2 μm in this case) was fairly small within each microscopic layer of the disk filter. For instance, for the 20-rice-husk filter, though 43.8% pores had sizes smaller than 2 μm, only 0.04% of water would flow through these small pores. Even for the 20%-starch filter, only 0.65% of water would pass through these small pores that result in the retention of bacteria within the thin layer.

TABLE 13
Percentage of <2 μm pores meausred by MIP and estmated flow
proportion passing through these small pores according to Hagen-Poiseuille
law for ceramic filters made of different combusible materials.

| Combutible | Cellulose fiber | | Starch | | Rice husk | |
|---|---|---|---|---|---|---|
| material | <2 μm | estimated flow | <2 μm | estimated flow | <2 μm | estimated flow |
| percentage | Pores | proportion | Pores | proprotion | Pores | proportion |
| (% wt) | (%) | (%) | (%) | (%) | (%) | (%) |
| 10 | 75.3 | 0.549 | 96.8 | 1.34 | 54.6 | 0.056 |
| 15 | 52.1 | 0.355 | 89.7 | 2.64 | 49.1 | 0.051 |
| 20 | 45.3 | 0.288 | 52.3 | 0.648 | 43.8 | 0.043 |
| 30 | 27.2 | 0.049 | 32.5 | 0.671 | — | — |

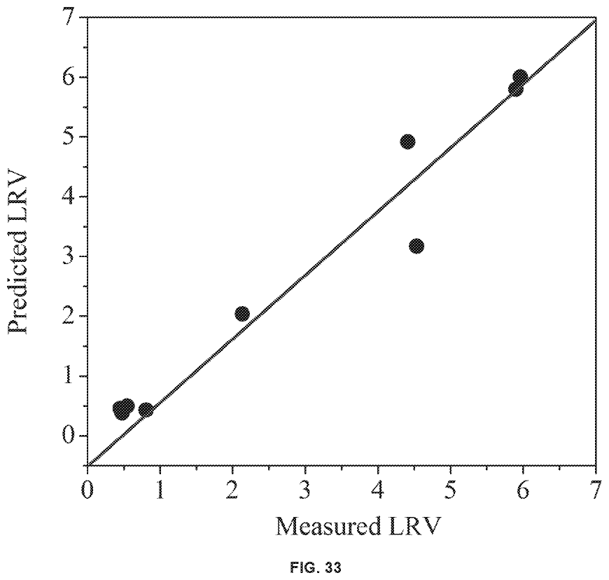

Based on the bacteria straining value within each microscopic layer, the overall bacterial removal efficiency of the ceramic disk filters was calculated using Equations 7 and 8. FIG. 33 showed the comparison of measured bacteria LRV versus predicated LRV based on the semi-quantitative model. The thickness of the microscopic layer was used as a fitting parameter with an optimum value of 6.67 μm (R²=0.95 of the regression). The E. coli removal efficiency values predicted by the semi-quantitative model compared favorably to the measured E. coli removal efficiency values, suggesting the model's potential usefulness in providing insights into the relationship between ceramic pore size distribution and microbial removal efficiency.

Our semi-quantitative model suggested the importance of pore size distribution in controlling the overall filter performance. For instance, both 15%-rice-husk and 15%-cellulose fiber filters had similar small pore percentages (49.1% and 52.1%, respectively; TABLE 13), but bacterial removal of the cellulose fiber filter was >3 LRV higher than that of the rice-husk filter, because of their different pore size distribution patterns that significant influenced the proportion of water flowing through the small pores. Specifically, the 15%-rice-husk filter had a bimodal pore size distribution pattern with two separated peaks and the water flow strongly preferred going through the pores around the larger peak (19.8-24.1 μm), according to Hagen-Poiseuille law (Equation 3). Thus, the proportion of water flowing through the small pores was only 0.05% within each microscopic layer, resulting in a relatively low overall bacterial removal efficiency. In contrast, due to the presence of medium (e.g., ˜10 μm) but not large (e.g., >20 μm) pores, the proportion of water flowing through small pores of the 15%-cellulose fiber filter was one order magnitude higher than that of the rice-husk filter, which caused a much higher overall bacterial removal. Meanwhile, although the starch filters had high bacterial removal efficiency because of their pore size distribution pattern with a single peak around size similar as bacterial cells, the lack of medium or large pores may significantly limit the water flow rate simultaneously. Our model analysis suggested that the combination of small pores, which led to microbial cell removal through straining, and medium pores, which assured adequate flow rate, may be favorable to achieve optimal ceramic filter designs that can balance effective microbial removal and adequate flow rate.

It is noted that, as previously suggested, removal of colloidal sized particles within porous media through physical straining could be more effective when many layered structures are stacked on top of each other (Xu et al., Environ. Sci. Technol. 2008, 42, 771-778; Xu et al., Water Resour. Res. 2006, 42; Xu et al., Water Resour. Res. 2009, 45). For ceramic disk filters, it is thus essential for the pores of various sizes to be interconnected in a random fashion: bigger pores (high flow rate) are connected to smaller pores where straining could occur. Advanced 3-dimensional (3-D) imaging techniques are powerful tools to provide pore network within porous media (Blunt et al., Adv. Water Resour. 2013, 51, 197-216; Guo et al., Fuel 2018, 230, 430-439). Such 3-D information for porous ceramic filters could enable the use of pore-scale mathematical and numeric models in future work to better understand the behavior (including straining) of microbial cells within ceramic filtration materials, and to provide guidance that can lead to improved water filtration ceramic disk filters.

### Example 13

### Practical Consideration and Implication

Results of the present study suggested the importance of combustible material on the performance of the ceramic filters. The improved performance of cellulose fiber filters may be attributed to the fibrous morphology of cellulose fiber that caused a unique pore size distribution pattern of the resulting filters. From SEM imaging analysis, cellulose fiber showed a tubular morphology with a median fiber diameter of 9.1 μm, which was clearly different from the spherical/oval shape of starch (with a median diameter of 15.2 μm) and the irregular shaped rice husk particles (with a median size of 21.4 μm) (FIG. 26). Compared to spherical or irregular particle shape, fibrous combustible material might promote the formation of continuous-phase and interconnected pores within the filter because of the relatively long tubular length, thus resulting in filters achieving simultaneous effective bacterial removal and high flow rate. In practice, the selection of combustible material may be determined by many factors (Rayner et al., J Water Sanit. Hyg. De. 2013, 3, 252-261; Klarman, Emory University, Atlanta, U.S. 2009). Fibrous materials other than cellulose fiber may also potentially be used as effective combustible materials based on their cost and local availability. It should be noted that in addition to combustible material, pore size distribution of ceramic filter can also be affected by other factors such as the clay composition (Oyanedel-Craver et al., Environ. Sci. Technol. 2008, 42, 927-933). Redart clay was selected as a model clay material in the present work to elucidate the role of combustible material, and future studies will be dedicated to investigate the performance of ceramic filters made of fibrous combustible material and locally source clay.

The batch experiments were conducted in a 20 mg/L As(III) solution mixed with 1.0 g/L adsorbent. The As(III) adsorption amount by La-modified blackbird clay ceramic granules was 9.6 mg/L while the As(III) adsorption amount by La-modified redart clay ceramic granules was 9.9 mg/g. The result suggests blackbird ceramic granules have a comparable As(M) adsorption capacity as La-modified redart clay ceramic. As(III) has no charge and is extremely difficult to remove, therefore this experiment shows that our materials can remove As(III) well (TABLE 14).

TABLE 14
Blackbird Clay data: Batch AS(III) Absorption.

| | |
|---|---|
| adsorbent (g/L) | As(III) adsorption |

| La-redart ceramic | blackbird clay | amount (mg/g) |
|---|---|---|
| 1 | 10 | 7.67 |
| 1 | 1 | 8.86 |
| 1 | 0 | 10.3 |

### Example 14

### Extremely Fast as Removal Using Granular La-Coated Ceramic Materials

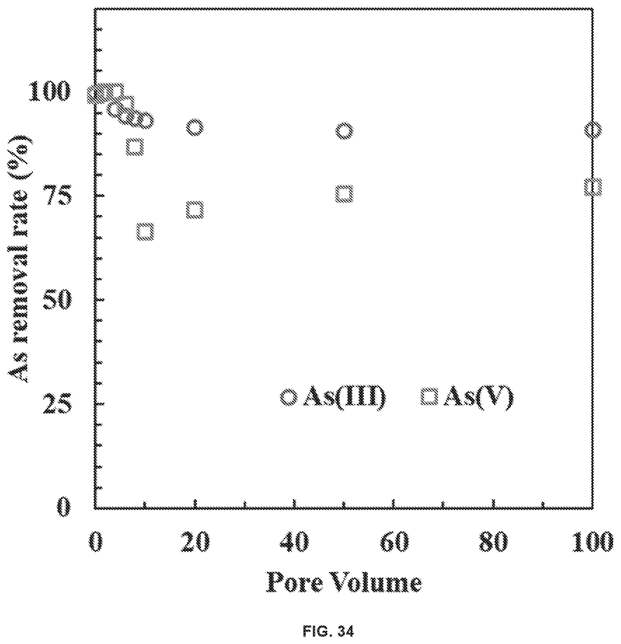

Granular ceramic filtration materials coated with La were packed into a column with a diameter of 1 cm and length of 15 cm. The pore volume of this packed granular column was ˜5.3 mL. As solutions (As(I) or As(V), 50 ppb) were injected into the packed column at an extremely fast flow rate (32 mL/min), which corresponds to a contact time of 10 seconds. The effluent samples were collected and the As concentrations were analyzed using ICP-MS. FIG. 34 shows the filtration results. On average, 94% of As(III) was removed through adsorption and 86% of As(V) was immobilized.

### Example 15

### Extremely Fast as Removal Using La-Coated Ceramic Disks

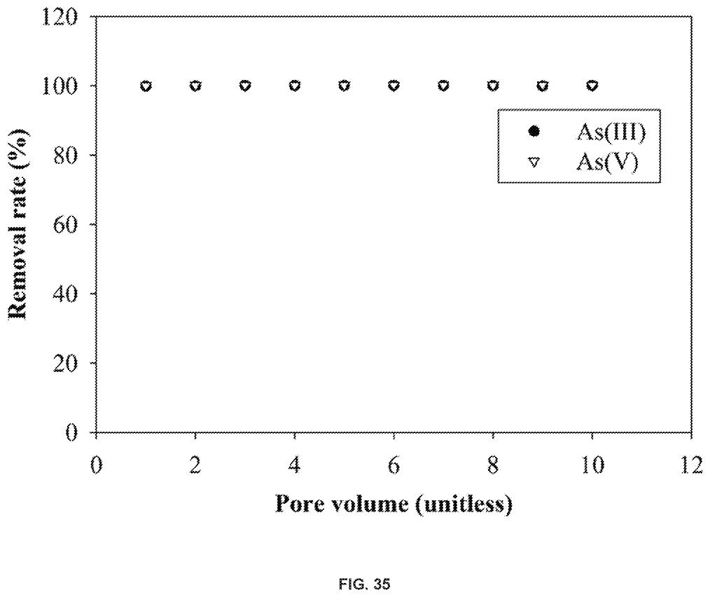

A ceramic disk measuring 1 inch in diameter and half inch in thickness was used. The flow rate was selected to produce a contact time of ˜40 seconds. As(III) or As(V) solutions that contain 50 ppb As were introduced to the disk and the filtrate was collected. ICP-MS was used to measure As concentrations. As shown in FIG. 35 100% of As was removed even under the extremely high flow rate and corresponding short contact time.

While several embodiments of the present invention have been described and illustrated herein, it is to be understood that the foregoing embodiments are presented by way of example only and that, within the scope of the appended claims and equivalents thereto, the invention may be practiced otherwise than as specifically described and claimed.

What is claimed is:

1. A filter material comprising:
a ceramic clay, the ceramic clay having an outer surface and a network of pores, the network of pores having a shape and a volume defined by combustion of cellulose fibers in a mixture of the cellulose fibers and a raw clay material.

2. The filter material of claim 1, wherein the mixture of the cellulose fibers and the raw clay material comprises 5-40 weight % of the cellulose fibers.

3. The filter material of claim 1 or 2, wherein the cellulose fibers have a size distribution of 0.1 μm to 100 μm in diameter.

4. The filter material of any of claims 1-3, wherein 10-60% of the pores have a diameter from >0 to 1 μm, 20-60% of the pores have a diameter from 1 to 10 μm, and 5-50% of the pores have a diameter from 10 to 100 μm.

5. The filter material of claim 4, wherein 50-60% of the cellulose fibers and/or the pores have a diameter from >0 to 1 μm, 30-40% of the cellulose fibers and/or the pores have a diameter from 1 to 10 μm, and 5-10% of the cellulose fibers and/or the pores have a diameter from 10 to 100 μm.

6. The filter material of claim 4, wherein 30-40% of the pores have a diameter from >0 to 1 μm, 50-60% of the pores have a diameter from 1 to 10 μm, and 10-20% of the pores have a diameter from 10 to 100 μm.

7. The filter material of claim 4, wherein 20-30% of the pores have a diameter from >0 to 1 μm, 50-60% of the pores have a diameter from 1 to 10 μm, and 20-30% of the pores have a diameter from 10 to 100 μm.

8. The filter material of claim 4, wherein 10-20% of the pores have a diameter from >0 to 1 μm, 35-45% of the pores have a diameter from 1 to 10 μm, and 40-50% of the pores have a diameter from 10 to 100 μm.

9. The filter material of claim 4, wherein 64-80% of the pores have a diameter less than 2 μm, 48-64% of the pores have a diameter less than 2 μm, 36-48% of the pores have a diameter less than 2 μm, or 20-36% of the pores have a diameter less than 2 μm.

10. The filter material of any of claims 1-9, wherein the cellulose fibers have a median fiber diameter of 1-10 μm.

11. The filter material of any of claims 1-10, wherein the cellulose fibers have a tubular shape.

12. The filter material of any of claims 1-11, wherein the cellulose fibers are recycled paper fibers.

13. The filter material of any of claims 1-12, wherein the pores have an average size of 0.9-9 μm.

14. The filter material of any of claims 1-13, wherein the ceramic clay has a porosity of 10% to 50%, 35-45%, 17-27%, or 10-20%.

15. The filter material of any of claims 1-14, wherein the ceramic clay is a ceramic of a fireclay, a kaolinite, an illite, a zeolite, diatomaceous earth, or a montmorillonite.

16. The filter material of any of claims 1-14, wherein the ceramic clay is a ceramic fireclay selected from a ceramic red art clay and a ceramic blackbird clay.

17. The filter material of any of claims 1-14, wherein the raw clay material comprises 30-90 wt. % SiO₂ and 2-40 wt. % Al₂O₃.

18. The filter material of any of claims 1-14, wherein the raw clay material comprises 30-70 wt. % SiO₂, 10-40 wt. % Al₂O₃, and 1-30 wt. % FeO₃.

19. The filter material of any of claims 1-18, wherein pores in the network of pores are substantially free of entrapped air and/or CO₂.

20. The filter material of any of claims 1-19, further comprising a coating disposed on the outer surface and within the network of pores of the ceramic clay, the coating comprising lanthanum.

21. The filter material of claim 20, wherein the coated ceramic clay has 0.5-25 wt. % of lanthanum.

22. The filter material of claim 20 or 21, wherein the coating is a coating heat treated on the ceramic clay at 100° C. to 800° C.

23. The filter material of any of claims 20-22, wherein the coating is a coating heat treated on the ceramic clay at 370° C. to 400° C.

24. The filter material of any of claims 20-23, wherein the coated ceramic clay displays FTIR peaks at about 3554, 1450, and/or 1300 cm¹.

25. The filter material of any of claims 20-24, wherein the coating comprises LaONO₃, La₂O₃, LaOOH, La₂(CO₃)₃, and/or La₂O₂CO₃.

26. The filter material of any of claims 20-25, wherein adsorption capacity for As(V) of the coated ceramic clay is 20-90 mg/g.

27. The filter material of any of claims 20-26, wherein adsorption capacity for Cr(VI) of the coated ceramic clay is 10-15 mg/g.

28. The filter material of any of claims 20-27, wherein adsorption capacity for As(III) of the coated ceramic clay is 2-25 mg/g.

29. The filter material of any of claims 1-28, wherein the ceramic clay is in the form of granules, a powders, disks, columns, or pots.

30. The filter material of claim 29, wherein the granules have an average diameter corresponding to 10 to 110 mesh.

31. A method of preparing the filter material of any of claims 1-18 comprising:
(a) homogenizing a mixture of raw clay material, cellulose fibers, and water;
(b) drying the homogenized mixture; and
(c) firing the dried, homogenized mixture so as to incinerate the cellulose fibers.

32. The method of claim 31, further comprising shaping the homogenized mixture into a disk, column, or pot.

33. The method of claim 31, further comprising grinding the fired mixture into granules or a powder.

34. A method of preparing the filter material of any of claims 20-30, comprising:
(a) treating an uncoated ceramic clay of any of claims 1-18 with a solution containing one or more lanthanum salts selected from the group consisting of La(NO₃)₃, LaCl₃, LaBr₃, LaI₃, LaF₃, La₂(SO₄)₃, LaPO₄, La₂(C₂O₄)₃, La₂O₃, LaOOH, La(OH)₃, La₂S₃, La(CH₃CO₂)₃, and LaAlO₃.
(b) heating the treated ceramic clay at 100° C. to 800° C.

35. The method of claim 34, further comprising cooling, rinsing, and drying the heat-treated ceramic clay.

36. A filter device comprising the filter material of any of claims 1-30 and a housing containing the filter material.

37. A method of removing a water contaminant from a water supply comprising contacting the water supply with the filter device of claim 36 to remove the water contaminant.

38. The method of claim 37, wherein the contaminant is a bacterium, a virus, As(III), As(V), or Cr(VI).
